(19) United States
(12) Patent Application Publication
(10) Pub. No.: US 2026/0274988 A1
(43) Pub. Date: Sep. 17, 2026

(54) ANTI-STEAP1 CAR

(71) Applicant: OSLO UNIVERSITETSSYKEHUS HF, Oslo (NO)

(72) Inventors: Jon AMUND KYTE, Hosle (NO); Yixin JIN, Oslo (NO)

(73) Assignee: OSLO UNIVERSITETSSYKEHUS HF, Oslo (NO)

(21) Appl. No.: 18/869,036

(22) PCT Filed: May 31, 2023
(86) PCT No.: PCT/EP2023/064599

§ 371 Date: Nov. 25, 2024

(30) Foreign Application Priority Data
Jun. 1, 2022 (GB) 2208119.4

(51) Int. Cl. C07K 16/40; A61K 40/11; A61K 40/31; A61K 40/42; A61P 35/00; A61P 35/04; C12N 5/0783

(52) U.S. Cl. CPC C07K 16/40; A61K 40/11; A61K 40/31; A61K 40/4244; A61P 35/00; A61P 35/04; C12N 5/0636; C12N 2510/00

(57) ABSTRACT

The present invention provides a nucleic acid molecule encoding a chimeric antigen receptor (CAR) directed against STEAP1, wherein said CAR comprises an antigen-binding domain comprising a V_{H} region and a V_{L} region, each comprising three CDR sequences, wherein: a) CDRs 1, 2 and 3 of the V_{H} region have the amino acid sequences of SEQ ID NOs: 14, 15 and 16 respectively; and b) CDRs 1, 2 and 3 of the V_{L} region have the amino acid sequences of SEQ ID NOs: 17, 18 and 19 respectively; and wherein one or more of said CDR sequences may optionally be modified by substitution, addition and/or deletion of 1 to 3 amino acids. A vector and an immune effector cell comprising the nucleic acid molecule of the invention, a composition, and a CAR are also provided herein.

### TECHNICAL FIELD OF THE INVENTION

The invention is related to the field of cell therapy. In particular, it relates to chimeric antigen receptors (CARs), nucleic acids encoding CARs, immune cells expressing them and their utility in medicine for treatment of prostate cancer.

### BACKGROUND

Prostate cancer is a common and lethal cancer with an unmet therapeutic need. Metastatic prostate cancer is generally considered incurable and current therapy only extends survival by months.

The six-transmembrane epithelial antigen of prostate 1 (STEAP1) is expressed in about 90% of prostate cancers, usually at high levels. Targeting of STEAP1 offers a new treatment option for prostate cancer. The present invention provides a CAR which targets STEAP1 and thus may be used in prostate cancer treatment. Expression of the CAR in an appropriate immune cell (e.g. T cell or NK cell) will redirect that cell to target cells expressing STEAP1.

In order to achieve a therapeutic CAR-immune cell, the cell needs to express the CAR in a sufficient amount in the cell membrane and the antigen binding domain of the CAR needs sufficient affinity and specificity for the target antigen. It has been observed that not every antibody antigen-binding domain is necessarily suitable to prepare a CAR, or effective in the context of a CAR.

CAR T cell therapy is an established therapy for haematological cancers. However, CAR T cell therapy for solid tumours provides a different set of obstacles compared to haematological malignancies. For example, the CAR T cells need to successfully find, enter and survive in the tumour. It can be expected that only a fraction of CAR T cells with in vitro activity will successfully migrate to tumour metastases in vivo and infiltrate the hostile tumour microenvironment of a solid tumour like prostate cancer. Furthermore, the CAR T cells will likely need to sustain their activity over time in order to provide a therapeutic effect.

It is therefore not trivial, but very desirable, to obtain novel CAR T or other immune cells able to provide a therapeutic effect on prostate cancer.

The present application demonstrates that T cells expressing the CAR of the invention provide an effective treatment for prostate cancer.

### SUMMARY OF INVENTION

Novel chimeric antigen receptors (CARs) are provided. When the CARs herein are expressed on the surface of immune cells, such immune cells are directed to STEAP1-positive prostate cancer cells as demonstrated by the in vitro and in vivo data provided in the Examples. The CARs are highly expressed in primary human T cells, both in cytotoxic T cells and T helper cells. The CAR T cells are polyfunctional, i.e. they produce significant amounts of IFNγ, TNFα and CD107α upon activation, which is considered important for clinical efficacy. The CAR T cells provide a cytotoxic effect against all STEAP1-positive cancer cell lines tested (both prostate cancer lines naturally expressing STEAP1 and negative cell lines transformed to express STEAP1). The cytotoxic effect of the CAR T cells is highly specific, as shown by comparison with non-transduced T cells from the same T cell donor. The in vivo CAR T activity in both subcutaneous and metastatic xenograft mouse models of prostate cancer is demonstrated. The CAR T cells infiltrated tumours and significantly inhibited tumour growth and extended survival in a STEAP1 dependent manner.

In a first aspect, the invention provides a nucleic acid molecule encoding a chimeric antigen receptor (CAR) directed against STEAP1, wherein said CAR comprises an antigen-binding domain comprising a V_{H} region and a V_{L} region, each comprising three CDR sequences, wherein:
a) CDRs 1, 2 and 3 of the V_{H} region have the amino acid sequences of SEQ ID NOs: 14, 15 and 16 respectively, and
b) CDRs 1, 2 and 3 of the V_{L} region have the amino acid sequences of SEQ ID NOs: 17, 18 and 19 respectively; and
wherein one or more of said CDR sequences may optionally be modified by substitution, addition and/or deletion of 1 to 3 amino acids.

In a second aspect, the invention provides a vector, preferably an expression vector or a cloning vector, comprising the nucleic acid molecule of the invention.

In a third aspect, the invention provides an immune effector cell comprising the nucleic acid molecule of the invention or the vector of the invention, wherein the immune effector cell expresses a CAR as defined in the first aspect of the invention at its surface.

In a fourth aspect, the invention provides a composition comprising the immune effector cell of the invention and a pharmaceutically acceptable carrier or excipient.

In a fifth aspect, the invention provides a CAR encoded by a nucleic acid molecule of the invention. Alternatively, according to this aspect the invention provides a CAR which comprises an antigen-binding domain comprising a V_{H} region and a V_{L} region, each comprising three CDR sequences, wherein:
a) CDRs 1, 2 and 3 of the V_{H} region have the amino acid sequences of SEQ ID NOs: 14, 15 and 16 respectively; and
b) CDRs 1, 2 and 3 of the V_{L} region have the amino acid sequences of SEQ ID NOs: 17, 18 and 19 respectively; and
wherein one or more of said CDR sequences may optionally be modified by substitution, addition and/or deletion of 1 to 3 amino acids.

When the CARs herein are expressed on the surface of immune cells, such immune cells may be used in medicine. In particular, said immune cells may be used in treatment of prostate cancer, including in particular in treatment of metastatic prostate cancer, or more particularly in the treatment of metastatic castration-resistant prostate cancer.

Accordingly, in a sixth aspect, the invention provides the immune effector cell of the invention, the composition of the invention, or the CAR of the invention, for use in therapy.

In a seventh aspect, the invention provides the immune effector cell of the invention, the composition of the invention, or the CAR of the invention, for use in the treatment of STEAP1-positive prostate cancer.

### BRIEF DESCRIPTION OF THE DRAWINGS

FIG. 1 outlines the schematic structure of the tested CARs. The CARs comprise, from N-terminus to C-terminus: an scFv comprising six CDRs, a hinge domain, a transmembrane domain, a costimulatory domain and an intracellular signaling domain. A shows the schematic structure of the CAR JK10; B shows the schematic structure of the CAR JK11. C shows the schematic structure of the CAR JK15. D shows the schematic structure of the CAR JK16.

FIG. 2 shows in vitro cytotoxicity of T cells expressing either JK10 or JK11, as measured by CD107a release upon contacting target cells. T cells were transduced with STEAP1-specific CARs (JK10 or JK11), or non-transduced (NT). The T cells were cultured with STEAP1-positive or STEAP1-negative target cells. Left panels: STEAP1-positive SupT1 cells (square and circle); STEAP1-negative SupT1 cells (triangle). Middle panels: LNCaP prostate cancer cells (STEAP1+). Right panels: 22Rv1 prostate cancer cells (STEAP1+). Cytotoxic activity was assessed by measurement of CD107a production in CAR+ cells compared with NT cells by flow cytometry. The assays demonstrated CD107a production from both JK10 and JK11 CAR T cells, specific for STEAP1+ targets, GeoMFI=Geometric Mean Fluorescence Intensity.

FIG. 3 shows that JK10 and JK11 CAR T cells specifically kill STEAP1 positive prostate cancer cells. T cells were transduced with JK10 (upper, red) or JK11 (bottom, red), or left non-transduced (blue). The T cells were cultured with STEAP1-positive prostate cancer cell lines LNCaP (left) or 22Rv1(right) for 16 hours (E:T=5:1). Caspase 3 cleavage in the target cells was measured by Flow cytometry. Caspase 3 cleavage is a measurement of cells undergoing apoptosis, i.e. in this case being killed by the CAR T cells.

FIG. 4 shows the IFNγ response by JK10 and JK11 CAR T cells to STEAP1+ tumour cells. T cells were transduced with STEAP1-specific CARs (JK10 or JK11), irrelevant CAR (antiCD19) or left non-transduced (NT). The T cells were co-cultured with various target cells for 16 hours. IFNγ production in the total CD3+ T-cell population was analysed by flow cytometry. In A, representative flow cytometry plots are displayed of IFNγ production in the total CD3+ T cell population of T cells co-cultured with LNCaP shGFP cells. B shows the combined data of IFNγ production in the total CD3+ T-cell populations following co-culture of the T cells with target cells (n=3). Explanation of target cells: LN shGFP is LNCaP treated with shRNA targeting GFP (shRNA negative control); LN sh8955 and LN sh9419 are LNCaP cells treated with two different shRNA knocking down STEAP1; C4-2B is a bone metastatic subline of C4-2 cells, which is an LNCaP-derived androgen insensitive cell line; C4-2B shGFP is C4-2B treated with shRNA targeting GFP; C4-2B sh8955 and C4-2B sh9419 are C4-2B cells treated with two different shRNA knocking down STEAP1; NALM6 (STEAP1-) is a B-cell precursor leukemia cell line, which is well established as target cells for anti-CD19 CAR T-cells.

FIG. 5 shows the TNF response of JK10 and JK11 CAR T cells to STEAP1+ tumour cells. T cells were transduced with STEAP1-specific CARs (JK10 or JK11), irrelevant CAR (anti-CD19) or left non-transduced (NT). The T cells were co-cultured with various target cells for 16 hours. TNF production in the total CD3+ T cell population was analysed by flow cytometry. In A, representative flow cytometry plots are displayed of TNF production in the total CD3+ T cell population of T cells co-cultured with LNCaP shGFP cells. B shows the combined data of TNF production in the total CD3+ T-cell populations following co-culture of the T cells with target cells (n=3). Explanation of target cells: See text for FIG. 4.

FIG. 6 shows that JK10 and JK11 CAR T cells specifically kill STEAP1+ tumour cells. Effector T cells were co-cultured with target cells for 16 hours, at which point caspase 3 cleavage in the target cells was measured. Caspase 3 cleavage is a measurement of cells undergoing apoptosis. A shows the gating strategy employed for calculating the percentage of apoptotic (cleaved caspase 3 positive) cells. B shows the percentage of cleaved caspase 3 positive cells. C depicts the cleaved caspase 3 data by GeoMFI. Caspase 3 cleavage in target cells was substantially higher when the target cells were co-cultured with STEAP1-specific CAR T cells than with non-transduced or control CAR T cells.

FIG. 7 shows the impact of JK10 CAR T cells and JK11 CAR T cells on the growth of prostate cancer cells in vivo (in a mouse xenograft model of prostate cancer). Immunodeficient NSG mice were injected on day 1 with ffLuc-labelled human prostate cancer cells (22Rv1; Steap-1+). On days 12 and 19 the mice received Steap1-specific CAR T cells (JK10 or JK11) or control non-transfected T cells (same donor). The effect was monitored by imagining (IVIS—detects luciferase signal) on day 11, 29 and 34. A significant difference in tumour load developed between JK10 and the control (p=0.004, Mann Whitney nonparametric test), with a mean 34-fold increase of tumour signal in the controls. JK11 showed a trend of reduction of cancer growth (5-fold reduction, not statistically significant). No adverse effects were observed in the mice.

FIG. 8 shows the results of JK11 CAR T therapy in a further murine NSG xenograft model of prostate cancer. In this instance, JK11 CAR T cells showed a statistically significant impact on cancer growth relative to the non-transduced control group (p=0.0009 at day 34) and the anti-CD19 control CAR T cell group (p=0.0188 at day 34; Mann Whitney nonparametric test). The mice were injected on day 1 with ffLuc-labelled human prostate cancer cells (Steap-1+). On day 9 all mice with a detectable tumour were selected for the assay, and received Steap1-specific CAR T cells or control non-transfected T cells (9-13 mice per group). A second injection with the same CAR T cells was given on day 14. In addition, the mice received rhIL-2 twice a week. The effect was monitored by imagining (IVIS—detects luciferase signal).

FIGS. 9 and 10 show further representations of the results of the in vivo experiment of FIG. 8, showing tumour size at day 34 based on IVIS analysis (FIG. 9) and caliper analysis on day 33 (FIG. 10).

FIG. 11 shows a bioluminescence image of the mice treated in the experiment of FIG. 8, taken at day 34 of the experiment. As shown, tumours had disappeared in 5 mice treated with the anti-STEAP1 CAR by this timepoint, and were clearly smaller on average in the treatment group than either control group.

FIG. 12 shows a survival curve for the mice treated in the experiment of FIG. 8. As shown, by day 60 all mice treated with NT T cells had to be euthanized and only three anti-CD19 CAR-treated mice were still alive. The mice treated with anti-STEAP1 CAR T cells had a significantly extended survival compared to both control groups, with 10 out of 12 mice still alive at this timepoint.

FIG. 13 shows the weight curves for the the in vivo experiment of FIGS. 8-10. Weight was monitored as a measurement of toxicity. No difference in weight development was detected between the JK11 CAR group and the controls (NT and anti-CD19 CAR).

FIG. 14 visualizes the results from Example 5 where NSG mice were injected i.v. into the tail vein with 10×10⁶ luciferase expressing 22Rv1 wildtype cells, or with 22Rv1 STEAP1 knock-out cells (C11). On day 19 and 26, the mice were treated i.v. with 10×10⁶ STEAP1 CAR T cells (N=5), or CD19 CAR T cells (N=5). 100 IU/g bodyweight rhIL-2 was given intraperitoneally twice a week. A) Tumour growth was measured once or twice a week by bioluminescence IVIS imaging. The data are presented as mean f SEM in each group of mice. B) Bioluminescence signals 33 days after tumour injection. The signal for each mouse is indicated. Statistical analyses were performed using Mann-Whitney U-test. C) Kaplan Meier plot showing extended survival for mice injected with wildtype 2Rv1 and treated with STEAP1 CAR T cells (dashed line), compared to CD19 CAR T cells (black line), and to mice injected with 22Rv1 knockout cells (C11; dotted lines). Statistical analysis was performed with log-rank test. The mice were euthanised when required by animal welfare guidelines, which generally corresponded to a total photon signal of 1×10¹⁰ (p/s). D-E) Tumours were fixed, paraffin-embedded and stained for IHC with anti-huCD3 (T cells) or QBen10 (CAR/RQR8 expressing cells). Figure D (aCD3) and E (aRQR8) show a representative staining from each group of mice, with 10× magnification. T cell infiltration (D) was only observed in the STEAP1 CAR T cell treated tumours and corresponded to CAR T cell infiltration (E).

FIG. 15 visualizes Real time monitoring of target killing by STEAP1 CAR T cells. The target cells 22Rv1 were transduced by lentivirus to express the nucleus-located protein GFP. One day before coculture with CAR-T cells, 22Rv1 target cells were seeded at 1×10⁵/well in 24-well plates and irradiated at 20Gy. Cryopreserved T cells were thawed and added to the target cells at the indicated E:T ratios. The plate was real-time monitored over 7 days and imaged by IncuCyte S3. (A) Mean GFP expression was determined by the IncuCyte S3 built-in software. (B) Representative images showing GPF+22Rv1 target cells at the start of the coculture and on day 6 (E:T=2.5:1).

### DETAILED DESCRIPTION

STEAP1 is highly expressed in prostate cancer cells and up-regulated in multiple other cancer forms (lung cancer, bladder cancer, Ewing sarcoma, breast cancer, pancreatic cancer, glioblastoma, ovarian cancer, leukemia, lymphoma, head and neck cancer). In normal tissues, STEAP1 is mainly expressed in the prostate, which is a dispensable organ.

The CAR of the invention is directed against STEAP1. The term “directed against STEAP1” is synonymous with “specific for STEAP1” or “anti-STEAP1”, that is it means simply that the CAR is capable of binding specifically to STEAP1. In particular, the antigen-binding domain of the CAR is capable of binding specifically to STEAP1 (more particularly when the CAR is expressed on the surface of an immune effector cell). Specific binding may be distinguished from non-specific binding to a non-target antigen (in this case an antigen other than STEAP1). Thus, an immune effector cell expressing the CAR according to the present invention is redirected to bind specifically to and exhibit cytotoxicity to (e.g. kill) a STEAP1-expressing target cell. Alternatively expressed, the immune effector cell is modified to redirect cytotoxicity towards target cells expressing STEAP1.

When the Chimeric Antigen Receptors (CARs) herein are expressed on the surface of immune cells the CARs are capable of binding to STEAP1 expressed on a target cell surface. Thus, such immune cells may be directed to prostate cancer cells and provide cytotoxic activity based on an accessible target epitope on STEAP1. As defined herein, CARs are receptors comprising an antigen-binding domain, typically derived from an antibody, and an intracellular signaling domain. Most commonly, CARs comprise, from N-terminus to C-terminus, an antigen binding domain, a hinge domain, a transmembrane domain and an intracellular signaling domain. As is known in the art, various intracellular co-stimulatory domains may also be included as part of the intracellular signaling domain. In other words, the intracellular signaling domain may comprise or consist of one or more parts or domains. A single chain Fv (scFv) is a preferred antigen-binding domain for use in the invention.

As used herein, an antigen binding domain (e.g. scFv) is a protein moiety able to bind an extracellular target epitope under physiological conditions, in particular physiological conditions in a tumour environment. The antigen-binding domains (e.g. scFvs) used in the CARs herein are able to bind to STEAP1. The antigen-binding domain of the CAR (e.g. the scFv) comprises two sequences; one variable domain from an antibody light chain (V_{L}) and one variable domain from an antibody heavy chain (V_{H}). A variety of anti-STEAP1 antibodies were screened for specific binding to prostate cancer cells, and the scFv used in the CARs disclosed herein was selected based on the antibody demonstrating the best binding.

In one embodiment, the scFv comprises or consists of, from N-terminus to C-terminus, V_{H}-linker-V_{L}. In another embodiment, the scFv comprises or consists of, from N-terminus to C-terminus, V_{L}-linker-V_{H}. The linker has a certain (sufficient) length in order to allow the V_{H} and V_{L} sequences to form a functional antigen-binding domain. In one embodiment, the linker comprises 10 to 30 amino acid residues. In one embodiment, the linker comprises 15 to 25 glycine and/or serine residues. Exemplary flexible linkers include glycine polymers (G)ₙ, glycine-serine polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the CARs described herein. In particular, the linker may comprise a number of “G₄S” repeats, in which the amino acid sequence motif “GGGGS” is repeated a certain number of times. In one embodiment, the linker is represented by the sequence GGGGSGGGGSGGGGSGGGGS (SEQ ID NO: 7).

Each V_{L} and V_{H} comprises three complementarity determining regions (CDRs) flanked by framework sequences. In the present invention, the antigen-binding domain (e.g. scFv) comprises:
a V_{L} comprising CDR1, CDR2 and CDR3 represented by KSSQSLLYRSNQKNYLA (SEQ ID NO: 17), WASTRES (SEQ ID NO: 18) and QQYYNYPRT (SEQ ID NO: 19), respectively;
and
a V_{H} comprising CDR1, CDR2 and CDR3 represented by GYSITSDYAWN (SEQ ID NO: 14), GYISNSGSTSYNPSLKSR (SEQ ID NO: 15) and ERNYDYDDYYYAMDY (SEQ ID NO: 16), respectively.
wherein one or more of said CDR sequences may optionally be modified by substitution, addition and/or deletion of 1 to 3 amino acids. The CDR sequences of SEQ ID NOs: 14-19 are derived from an anti-STEAP1 antibody known as Oslo-1.

In an embodiment the CDR sequences are modified by substitution of 1-3 amino acids.

Preferably, any substitution of an amino acid within a CDR sequence is a conservative amino acid substitution. The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including amino acids with basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus a conservative amino acid substitution may be considered to be a substitution in which a particular amino acid residue is substituted for a different amino acid residue in the same family.

In one embodiment, the antigen-binding domain (e.g. scFv) comprises

a V_{H} comprising CDR1, CDR2 and CDR3 having the amino acid sequences of SEQ ID NOs: 14, 15 and 16, respectively;
and
   a V_{L} comprising CDR1, CDR2 and CDR3 having the amino acid sequences of SEQ ID NOs: 17, 18 and 19 respectively. Thus in a preferred embodiment the antigen-binding domain comprises the unmodified Oslo-1 CDR sequences.

The Oslo-1 antibody is a known antibody (see e.g. WO 2008/052187 and WO 2018/184966). WO 2018/184966 discloses a number of variants of the Oslo-1 antibody, comprising up to 2 amino acid substitutions in the VHCDR3 sequence relative to the native sequence (SEQ ID NO: 16). In a particular embodiment of the invention, the VHCDR3 sequence comprises up to two amino acid substitutions relative to the native VHCDR3 sequence of SEQ ID NO: 16, and all other CDR sequences are unmodified relative to the native sequences. In other words, CDRs 1 and 2 of the V_{H} region have the amino acid sequences of SEQ ID NOs: 14 and 15 respectively; CDRs 1, 2 and 3 of the V₁, region have the amino acid sequences of SEQ ID NOs: 17, 18 and 19 respectively; and CDR3 of the V_{H} region has the amino acid sequence of SEQ ID NO: 16, or an amino acid sequence comprising up to 2 amino acid substitutions relative to SEQ ID NO: 16.

The variant VHCDR3 sequences disclosed in WO 2018/184966 have the amino acid sequences set forth in SEQ ID NOs: 20, 21 and 22. SEQ ID NO: 20 contains an Asp=>Glu substitution at position 7, relative to SEQ ID NO: 16; SEQ ID NO: 21 contains an Asp=>Glu substitution at position 8, relative to SEQ ID NO: 16; SEQ ID NO: 22 contains two Asp=>Glu substitutions at positions 7 and 8 relative to SEQ ID NO: 16. These VHCDR3 sequences may be used in the antigen-binding domain of the CAR of the invention. Thus in a particular embodiment, CDRs 1 and 2 of the V_{H} region have the amino acid sequences of SEQ ID NOs: 14 and 15 respectively; CDRs 1, 2 and 3 of the V_{L} region have the amino acid sequences of SEQ ID NOs: 17, 18 and 19 respectively; and CDR3 of the V_{H} sequence has the amino acid sequence of SEQ ID NO: 20, SEQ ID NO: 21 or SEQ ID NO: 22.

In one embodiment, the antigen-binding domain comprises a V_{H} comprising the amino acid sequence set forth in SEQ ID NO: 1, or a sequence with at least 80, 85, 90 or 95% sequence identity to SEQ ID NO: 1; and a V_{L} comprising the amino acid sequence set forth in SEQ ID NO: 2, or a sequence with at least 80, 85, 90 or 95% sequence identity to SEQ ID NO 2. In particular, it is preferred that the antigen-binding domain is a scFv comprising the aforementioned V_{H} and V_{L} sequences. Variation relative to the amino acid sequences of SEQ ID NOs: 1 and 2 may be in the form of amino acid substitutions, insertions or deletions, but preferably is in the form of conservative substitution of amino acid residues. While variation of the V_{H} and V_{L} sequences is thus permitted, this is of course subject to the proviso that the CDR sequences remain as defined above, and in one particular embodiment to the proviso that the CDR sequences are unaltered.

The framework regions of the V_{L} and V_{H} sequences may accordingly be modified by one or more amino acid substitutions, additions or deletions. Substitutions of amino acid residues may be tolerated better than deletions or additions of amino acid residues. The substitutions may be conservative substitutions as discussed above.

In a preferred embodiment, the scFv comprises the V_{H} and V_{L} sequences described above, joined by the linker of SEQ ID NO: 7. The CAR of the invention may thus comprise an scFv comprising or consisting of the amino acid sequence set forth in SEQ ID NO: 23, or an amino acid sequence having at least 90 or 95% sequence identity thereto. The scFv comprises the V_{H} of SEQ ID NO: 1 at its N-terminus and the V_{L} of SEQ ID NO: 2 at its C-terminus, separated by the linker of SEQ ID NO: 7.

The CAR of the invention generally comprises a hinge domain. The hinge domain connects the antigen-binding domain (e.g. scFv) to the transmembrane domain. In particular, the CARs in the present disclosure may comprise a human CD8α hinge (SEQ ID NO: 9) or an IgG-based hinge, e.g. a native IgG hinge or a variant thereof, for example a deletion variant of an IgG hinge comprising a deletion in the hinge sequence. Preferred IgG-based hinges include the native IgG hinge (from human IgG1), which has the sequence set forth in SEQ ID NO: 30, and the IgG-based hinge disclosed in Hombach et al., Gene Therapy 17: 1206-1213 (2010), known as the IgGΔ hinge, which has the amino acid sequence set forth in SEQ ID NO: 3. In the IgGΔ hinge, the PELLGG and ISR sequence motifs are disrupted by amino acid substitution and deletion such that their sequences are instead PPVAG and IAR, respectively. Disruption of these motifs prevents cross-activation of FcγR+ cells. The CAR may thus comprise the CD8α hinge domain, comprising or consisting of the amino acid sequence of SEQ ID NO: 9, or an amino acid sequence having at least 95% sequence identity thereto: the IgG hinge domain, comprising or consisting of the amino acid sequence of SEQ ID NO: 30, or an amino acid sequence having at least 95% sequence identity thereto; or the IgGΔ hinge domain, comprising or consisting of the amino acid sequence of SEQ ID NO: 3, or an amino acid sequence having at least 95% sequence identity thereto. Other variants of the native IgG hinge may also be used. In particular, IgG hinge variants comprising one or more, but not all, of the sequence modifications present in the IgGΔ hinge relative to the native IgG hinge may be used. Other suitable hinge domains include the hinge domains of human CD4, CD28 and CD7.

In particular, the CARs in the present disclosure may comprise an scFv as mentioned above connected to a CD8α hinge, wherein the CAR further comprises a transmembrane domain and wherein the intracellular signaling domain comprises or consists of one costimulatory domain from CD28, 4-1BB or OX40 and a CD3ζ signaling domain.

Alternatively, the CARs in the present disclosure may comprise an scFv as mentioned above connected to an IgG hinge, wherein the CAR further comprises a transmembrane domain and wherein the intracellular signaling domain comprises or consists of one costimulatory domain from CD28, 4-1BB or OX40 and a CD3ζ signaling domain.

The hinge domain may affect the steric conformation of the scFv. This may in turn affect the ability of the CAR to bind the target epitope and subsequently trigger signaling in an immune cell. If the target epitope is located too far from the cell membrane of the target cell or if the target epitope is otherwise hidden, the immune cell expressing the CAR may not be efficient. Accordingly, it is preferred that the target epitope is sufficiently accessible for immune cells expressing the CARs. It has been found that an IgGΔ hinge (represented by SEQ ID NO: 3) in the CARs disclosed herein allows the antigen binding domain to bind to the target epitope and allows activation of the immune cell expressing it. Other IgG hinges may work as well provided they allow a similar steric conformation of the antigen binding domain. In particular, such hinges may comprise approximately the same number of amino acid residues as the IgGΔ hinge represented by SEQ ID NO: 3. It is preferred that the IgG hinge is modified to reduce its affinity for Fc-receptors. This may avoid off-target stimulation of the CAR T cells. One example of such a modified hinge is represented by SEQ ID NO: 3. In particular, the IgGΔ hinge can be represented by SEQ ID NO: 3 or sequences with at least 90% sequence identity thereto.

It has been found that a human CD8α hinge (represented by SEQ ID NO: 9) in the CARs disclosed herein allows the antigen binding domain to bind to the target epitope and allows activation of the immune cell expressing it. Other CD8α hinges may work as well provided they allow a similar steric conformation of the antigen binding domain. In particular, such hinges may comprise approximately the same number of amino acid residues as the CD8α hinge represented by SEQ ID NO: 9. In particular, the CD8α hinge can be represented by SEQ ID NO: 9 or sequences with at least 90% sequence identity thereto.

Sequence identity may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet., 16, (6) pp276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32(5):1792-1797, 2004) may be used for multiple sequence alignments, though any other appropriate programme may be used. Another suitable alignment programme is BLAST, using the blastp algorithm for protein alignments and the blastn algorithm for nucleic acid alignments. Whether the alignment is pairwise or multiple, it must be performed globally (i.e. across the entirety of the reference sequence) rather than locally.

Sequence alignments and % identity calculations may be determined using for instance standard Clustal Omega parameters: matrix Gonnet, gap opening penalty 6, gap extension penalty 1. Alternatively the standard EMBOSS Needle parameters may be used: matrix BLOSUM62, gap opening penalty 10, gap extension penalty 0.5. Any other suitable parameters may alternatively be used.

The CAR of the invention comprises a transmembrane domain. The transmembrane domain connects the extracellular domains to an intracellular signaling domain. The antigen-binding domain (e.g. scFv) and hinge are the extracellular domains. As used herein, “transmembrane domain”, means the part of the CAR which is embedded in the cell membrane when expressed by an immune effector cell. Suitable transmembrane domains are well known for skilled persons.

The transmembrane domain may be based on or derived from the transmembrane domain of any transmembrane protein. Typically it may be, or may be derived from, a transmembrane domain from CD8a, CD28, CD4, CD3ζ, CD45, CD9, CD16, CD22, CD33, CD64, CD80, CD86, CD134, CD137, or CD154, preferably from a human version of these proteins. In one embodiment, the transmembrane domain may be, or may be derived from, a transmembrane domain from CD8α, CD28, CD4, or CD3ζ, preferably from human CD8α, CD28, CD4, or CD3ζ. In another embodiment the transmembrane domain may be synthetic in which case it would comprise predominantly hydrophobic residues such as leucine and valine.

In particular, transmembrane domains from CD8α and CD28 can be used in the present invention. The transmembrane domain is believed to convey a signal into immune cells upon binding of a target by the antigen binding domain. It has been found that the CD28 transmembrane domain represented by SEQ ID NO: 4 allows signaling into an immune cell upon binding of a target. It has also been found that the CD8α transmembrane domain represented by SEQ ID NO: 10 allows signaling into an immune cell upon binding of a target. Thus, in a particular embodiment, the transmembrane domain of the CAR of the invention is the CD28 transmembrane domain, comprising or consisting of the amino acid sequence of SEQ ID NO: 4, or an amino acid sequence having at least 95% sequence identity thereto. In another embodiment, the transmembrane domain of the CAR of the invention is the CD8α transmembrane domain, comprising or consisting of the amino acid sequence of SEQ ID NO: 10, or an amino acid sequence having at least 95% sequence identity thereto.

In a particular embodiment, the CAR of the invention comprises an IgG hinge and a CD28 transmembrane domain. In another embodiment, the CAR of the invention comprises a CD8α hinge and a CD8α transmembrane domain.

The CARs of the invention also comprise an intracellular signaling domain. The “intracellular signaling domain” refers to the part of the CAR located inside the immune cell when the CAR is expressed in the cell membrane. These domains participate in conveying the signal upon binding of the target. In particular, the intracellular signalling domain refers to the part of the CAR protein that participates in transducing the message of effective CAR binding to a target antigen into the interior of the immune effector cell to elicit effector cell function, e.g. activation, cytokine production, proliferation and/or cytotoxic activity, including the release of cytotoxic factors to the CAR-bound target cell, or other cellular responses elicited with antigen binding to the extracellular CAR domain. The term “effector function” refers to a specialized function of the cell. Effector function of the T-cell, for example, may be cytolytic activity or help or activity including the secretion of a cytokine. Thus, the term “intracellular signalling domain” refers to the portion of a protein which transduces the effector function signal and that directs the cell to perform a specialized function.

While an entire intracellular signalling domain can be employed, in many cases it is not necessary to use the entire domain. To the extent that a truncated portion of an intracellular signalling domain is used, such a truncated portion may be used in place of the entire domain as long as it transduces the effector function signal. The term intracellular signalling domain is meant to include any truncated portion of the intracellular signalling domain sufficient to transduce effector function signal. The intracellular signalling domain is also known as the “signal transduction domain”. A variety of signaling domains are known, and they can be combined and tailored to fit the endogenous signaling machinery in the immune cells.

In one embodiment the intracellular signaling domain comprises a “signal 1” domain, such as the signaling domain from CD3ζ, FcR-γ, CD3ε etc. In general, it is believed that “signal 1” domains (e.g. the CD3ζ signaling domain represented by SEQ ID NO: 6) convey a signal upon antigen binding. It is particularly preferred that the CAR of the invention comprises the CD3ζ signaling domain, comprising or consisting of the amino acid sequence of SEQ ID NO: 6, or an amino acid sequence having at least 95% sequence identity thereto.

In another embodiment, the intracellular signaling domain further comprises a costimulatory domain. Such domains are well known and often referred to as “signal 2” domains, and they are believed to, subsequently to “signal 1” domains, convey a signal via costimulatory molecules. The “signal 2” is important for the maintenance of the signal and the survival of the cells. If absent, like in first generation CARs, the redirected cell may be efficient in killing and in early cytokine release, but will often become exhausted over time.

The term “co-stimulatory signalling domain” or “co-stimulatory domain”, refers to the portion of the CAR comprising the intracellular domain of a co-stimulatory molecule. Co-stimulatory molecules are cell surface molecules other than antigen receptors or Fc receptors that provide a second signal required for efficient activation and function of an immune effector cell (e.g. a T-cell) upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83, more particularly the intracellular domains of such molecules. Preferably the molecules are human. Accordingly, while exemplary or preferred co-stimulatory domains are derived from 4-1BB, CD28 or OX40 (CD134), other co-stimulatory domains are contemplated for use with the CARs described herein. The co-stimulatory domains may be used singly or in combination (i.e. one or more co-stimulatory domains may be included in a single CAR. The inclusion of one or more co-stimulatory signalling domains may enhance the efficacy and expansion of immune effector cells expressing the CARs. The intracellular signalling and co-stimulatory signalling domains may be linked in any order in tandem to the carboxyl terminus of the transmembrane domain.

As noted above, preferred examples of such “signal 2”, or co-stimulatory, domains include the 4-1BB costimulatory domain (SEQ ID NO: 11) and the CD28 costimulatory domain (SEQ ID NO: 5).

In particular, it has been found that cytotoxic immune cells expressing the CARs herein provide an in vivo effect in prostate cancer models when the intracellular signaling domain comprises or consists of a costimulatory domain (SEQ ID NO: 5 OR SEQ ID NO: 11) and a CD3ζ signaling domain (SEQ ID NO: 6). It is thus preferred that the intracellular signaling domain comprises the CD3ζ signaling domain (i.e. the amino acid sequence of SEQ ID NO: 6, or an amino acid sequence having at least 95% sequence identity thereto) and a co-stimulatory domain. In a particular embodiment, the intracellular signaling domain comprises the CD3ζ signaling domain (i.e. the amino acid sequence of SEQ ID NO: 6, or an amino acid sequence having at least 95% sequence identity thereto) and the 4-1BB co-stimulatory domain, comprising or consisting of the amino acid sequence of SEQ ID NO: 11, or an amino acid sequence having at least 95% sequence identity thereto. In another embodiment, the intracellular signaling domain comprises the CD3ζ signaling domain (i.e. the amino acid sequence of SEQ ID NO: 6, or an amino acid sequence having at least 95% sequence identity thereto) and the CD28 costimulatory domain, comprising or consisting of the amino acid sequence of SEQ ID NO: 5, or an amino acid sequence having at least 95% sequence identity thereto.

For efficient expression of the claimed CARs in immune cells, a conventional leader peptide (also known as signal peptide or L-chain) may be provided to drive localisation of the CAR to the cell membrane. Such a sequence will generally be provided at the N-terminal end of the molecule (construct) and may function to, co-translationally or post-translationally, direct transfer of the molecule to the membrane (i.e. the plasma membrane of an immune effector cell). The signal sequence may be linked directly or indirectly (e.g. via a linker sequence) to the antigen-binding domain of the CAR molecule/construct. Preferably, the signal sequence is linked directly to the N-terminus of the antigen-binding domain. The leader peptide is believed to be trimmed off following delivery of the CAR to the membrane, and so will likely not be present in the functional CAR in the cell membrane. It has been found that the nucleic acids encoding the CARs herein are successfully transcribed and the CARs are successfully localised to the cell membrane when the leader peptide is represented by SEQ ID NO: 8. Thus, in an embodiment, the CAR of the invention, as encoded or translated, comprises an N-terminal signal sequence. In a particular embodiment, the N-terminal signal sequence comprises or consists of SEQ ID NO: 8. Alternatively expressed, the nucleic acid molecule may comprise a nucleotide sequence encoding a leader peptide as described herein.

As shown in the Examples, a number of functional anti-STEAP1 CARs have been developed by the inventors. One of these is the CAR JK10. JK10 comprises, from N-terminus to C-terminus, the scFv of SEQ ID NO: 23, the IgGΔ hinge of SEQ ID NO: 3, the CD28 transmembrane domain of SEQ ID NO: 4, the CD28 costimulatory domain of SEQ ID NO: 5 and the CD3ζ signaling domain of SEQ ID NO: 6. JK10 has the amino acid sequence set forth in SEQ ID NO: 24. In an embodiment of the invention, the CAR is the JK10 CAR or a variant thereof. That is to say, the CAR comprises the amino acid sequence of SEQ ID NO: 24, or an amino acid sequence having at least 95% sequence identity thereto.

Another anti-STEAP1 CAR shown in the Examples is the CAR JK11. JK11 comprises, from N-terminus to C-terminus, the scFv of SEQ ID NO: 23, the CD8α hinge of SEQ ID NO: 9, the CD8α transmembrane domain of SEQ ID NO: 10, the 4-1BB co-stimulatory domain of SEQ ID NO: 11 and the CD3ζ signaling domain of SEQ ID NO: 6. JK11 has the amino acid sequence set forth in SEQ ID NO: 25. In an embodiment of the invention, the CAR is the JK11 CAR or a variant thereof. That is to say, the CAR comprises the amino acid sequence of SEQ ID NO: 25, or an amino acid sequence having at least 95% sequence identity thereto.

Another anti-STEAP1 CAR is the CAR JK15. JK15 comprises, from N-terminus to C-terminus, the scFv of SEQ ID NO: 23, the IgGΔ hinge of SEQ ID NO: 3, the CD28 transmembrane domain of SEQ ID NO: 4, the 4-1BB co-stimulatory domain of SEQ ID NO: 11 and the CD3ζ signaling domain of SEQ ID NO: 6. JK15 has the amino acid sequence set forth in SEQ ID NO: 26. In an embodiment of the invention, the CAR is the JK15 CAR or a variant thereof. That is to say, the CAR comprises the amino acid sequence of SEQ ID NO: 26, or an amino acid sequence having at least 95% sequence identity thereto.

Another anti-STEAP1 CAR is the CAR JK16. JK16 comprises, from N-terminus to C-terminus, the scFv of SEQ ID NO: 23, the CD8α hinge of SEQ ID NO: 9, the CD8α transmembrane domain of SEQ ID NO: 10, the CD28 costimulatory domain of SEQ ID NO: 5 and the CD3ζ signaling domain of SEQ ID NO: 6. JK16 has the amino acid sequence set forth in SEQ ID NO: 27. In an embodiment of the invention, the CAR is the JK16 CAR or a variant thereof. That is to say, the CAR comprises the amino acid sequence of SEQ ID NO: 27, or an amino acid sequence having at least 95% sequence identity thereto.

CAR T-cells expressing JK10, JK11, JK15 or JK16 all provided cytotoxic activity against LNCap cells in a caspase assay.

As noted above, the CARs of the invention are generally encoded with a leader peptide (signal peptide) in order to target them to the plasma membrane of the immune effector cell in which they are expressed. As detailed above, a suitable leader sequence for inclusion in the CARs of the invention is that of SEQ ID NO: 8. The JK10 CAR comprising the leader sequence of SEQ ID NO: 8 has the amino acid sequence set forth in SEQ ID NO: 12, the JK11 CAR comprising the leader sequence of SEQ ID NO: 8 has the amino acid sequence set forth in SEQ ID NO: 13, the JK15 CAR comprising the leader sequence of SEQ ID NO: 8 has the amino acid sequence set forth in SEQ ID NO: 28 and the JK16 CAR comprising the leader sequence of SEQ ID NO: 8 has the amino acid sequence set forth in SEQ ID NO: 29. In particular embodiments of the invention, the nucleic acid molecule encodes a CAR comprising or consisting of the amino acid sequence set forth in SEQ ID NO: 12, 13, 28 or 29, or an amino acid sequence having at least 95% sequence identity thereto.

The present disclosure provides CAR polypeptides, and fragments thereof. The terms “polypeptide” and “protein” are used interchangeably and mean a polymer of amino acids not limited to any particular length. The term does not exclude modifications such as myristylation, sulfation, glycosylation, phosphorylation and addition or deletion of signal sequences. The terms “polypeptide” and “protein” mean one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass the CARs of the present disclosure, or sequences that have deletions from, additions to, and/or substitutions of one or more amino acid of a CAR as disclosed herein.

As is clear from the above, the various domains of the CAR may comprise one or more amino acid sequence modifications with respect to the native sequences of the molecules from which they are derived. For example, it may be desirable to improve the binding affinity and/or other biological properties of the CAR. For example, amino acid sequence variants of a CAR, or binding domain, or a stimulatory signalling domain thereof, may be prepared by introducing appropriate nucleotide changes into a polynucleotide that encodes the CAR, or a domain thereof. Such modifications include, for example, deletions from, and/or insertions into and/or substitutions of, residues within the amino acid sequences of the CAR. Any combination of deletion, insertion, and substitution may be made to arrive at the final CAR, provided that the final construct possesses the desired characteristics, such as specific binding to STEAP1 by the binding domain, or increased signalling by the intracellular signalling domain and/or co-stimulatory domain. The amino acid changes also may alter post-translational processes of the CAR, such as changing the number or position of glycosylation sites. Any of the variations and modifications described above may be included in the CARs of the present invention.

The nucleic acid molecule of the invention may be an isolated nucleic acid molecule and may further include DNA or RNA or chemical derivatives of DNA or RNA, including molecules having a radioactive isotope or a chemical adduct such as a fluorophore, chromophore or biotin (“label”). Thus the nucleic acid may comprise modified nucleotides. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “nucleic acid molecule” specifically includes single- and double-stranded forms of DNA and RNA.

Methods for modifying nucleotide sequences to introduce changes to the amino acid sequences of the various domains are well known in the art, e.g. methods of mutagenesis, such as site-specific mutagenesis, may be employed.

Likewise methods for preparing a nucleic acid molecule encoding the CAR are also well known e.g. conventional polymerase chain reaction (PCR) cloning techniques can be used to construct the nucleic acid molecule. The nucleic acid molecule can be cloned into a general purpose cloning vector such as pENT (Gateway), pUC19, pBR322, pBluescript vectors (Stratagene Inc.) or pCR TOPO® from Invitrogen Inc. The resultant nucleic acid construct (recombinant vector) carrying the nucleic acid molecule encoding the CAR can then be sub-cloned into expression vectors or viral vectors for protein expression, e.g. in mammalian cells. This may be for preparation of the CAR protein, or for expression in immune effector cells, e.g. in human T-cells or in NK cells or cell lines. Further the nucleic acid may be introduced into mRNA expression vectors for production of mRNA encoding the CAR. The mRNA may then be transferred into immune effector cells.

Thus the nucleic acids of the invention can be provided in the context of a vector. The vector may be an RNA (e.g. mRNA) or DNA vector (e.g. expression vectors, retroviral vectors etc.). The vector may in particular be a cloning vector or an expression vector. Expression vectors can contain a variety of control sequences, which refer to nucleic acid sequences necessary for the transcription and possibly translation of an operatively linked coding sequence in a particular host cell. In addition to control sequences that govern transcription and translation, vectors may contain additional nucleic acid sequences that serve other functions, including for example for replication, selectable markers etc. Such control sequences, and other functional sequences, are well-known in the art.

For cloning of the nucleic acid molecule the vector may be introduced into a host cell (e.g. an isolated host cell) and such “production host cells” containing a cloning vector of the invention may form a further aspect of the invention. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheniformis, Pseudomonas such as P. aeruginosa, and Streptomyces. A production host cell may alternatively contain an mRNA expression vector comprising the nucleic acid molecule.

The nucleic acid molecules or vectors are introduced into a host cell (e.g. a production host cell or an immune effector cell) using transfection and/or transduction techniques known in the art. As used herein, the terms, “transfection,” and, “transduction,” refer to the processes by which an exogenous nucleic acid sequence is introduced into a host cell. The nucleic acid may be integrated into the host cell DNA or may be maintained extra-chromosomally. The nucleic acid may be maintained transiently or may be stably introduced. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection. In certain embodiments, retroviral vectors are transduced by packaging the vectors into viral particles or virions prior to contact with a cell.

An “immune effector cell,” is any cell of the immune system that has one or more effector functions (e.g. cytotoxic cell killing activity, secretion of cytokines, induction of ADCC and/or CDC). Representative immune effector cells thus include T lymphocytes, in particular cytotoxic T cells (CTLs; CD8+ T cells) and helper T-cells (HTLs; CD4+ T cells). Other populations of T cells are also useful herein, for example naive T cells and memory T cells. Other immune effector cells include NK cells, NKT cells, neutrophils, and macrophages. Immune effector cells also include progenitors of effector cells, wherein such progenitor cells can be induced to differentiate into an immune effector cells in vivo or in vitro. As described further below, the cells may be primary cells or cell lines.

T cells, particularly CD8+ T cells, and NK cells represent preferred immune effector cells according to the invention. The invention provides an immune effector cell comprising a nucleic acid molecule or vector of the invention and expressing a CAR of the invention at its surface.

The term “NK cell” refers to a large granular lymphocyte, being a cytotoxic lymphocyte derived from the common lymphoid progenitor which does not naturally comprise an antigen-specific receptor (e.g. a T-cell receptor or a B-cell receptor). NK cells may be differentiated by their CD3−, CD56+ phenotype. The term as used herein thus includes any known NK cell or any NK-like cell or any cell having the characteristics of an NK cell. Thus primary NK cells may be used or in an alternative embodiment, a NK cell known in the art that has previously been isolated and cultured may be used. Thus an NK cell-line may be used. A number of different NK cells are known and reported in the literature and any of these could be used, or a cell-line may be prepared from a primary NK cell, for example by viral transformation (Vogel et al. 2014, Leukemia 28:192-195). Suitable NK cell lines include (but are by no means limited to), in addition to NK-92, the NK-YS, NK-YT, MOTN-1, NKL, KHYG-1, HANK-1, or NKG cell lines. Similarly, a T cell of the invention may be a primary T cell, or a T cell line. Preferably, the immune effector cell of the invention is a human NK cell or a human T cell (e.g. a human CD8+ T cell or a human CD4+ T cell). Such cells are particularly suitable for treatment of human patients.

The immune cells used to express the CARs herein may be isolated from a patient or a compatible donor by leukapheresis or other suitable methods. As noted above, such primary cells may in particular be T cells or NK cells. In particular, autologous T cells (both cytotoxic T cells. T helper cells or mixtures of these) may be transduced with nucleic acids encoding the CARs before a pharmaceutical composition comprising the cells is administered to the patient.

The pharmaceutical compositions herein can be any composition suitable for administration of therapeutic cells to a patient. The most common administration route for CAR T cells is intravenous administration. Accordingly, said pharmaceutical compositions may for example be sterile aqueous solutions with a neutral pH. For example, a patient's peripheral blood mononuclear cells may be obtained via a standard leukapheresis procedure. The mononuclear cells may be enriched for T cells, before transducing them with a lentiviral vector or mRNA encoding the CARs. Said cells may then be activated with anti-CD3/CD28 antibody coated beads. The transduced T cells may be expanded in cell culture, washed, and formulated into a sterile suspension, which can be cryopreserved. If so, the product is thawed prior to administration.

Pharmaceutical compositions of the present invention may thus comprise a CAR-expressing immune effector cell population, such as T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins: polypeptides or amino acids such as glycine; antioxidants; chelating agents such as EDTA or glutathione; adjuvants (e.g. aluminium hydroxide); and preservatives.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono- or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

As detailed above, novel chimeric antigen receptors (CARs) are provided herein. When the CARs herein are expressed on the surface of immune cells, such immune cells may be used in medicine. In particular, said immune cells may be used in the treatment of prostate cancer. In one embodiment, said immune cells may be used in the treatment of metastatic prostate cancer. In one embodiment, said immune cells may be used in the treatment of castration-resistant prostate cancer. In another embodiment, said immune cells may be used in the treatment of metastatic castration-resistant prostate cancer. The animal data provided herein, indicate that STEAP1 CAR T cells retained their anti-tumour activity in vivo, and exerted tumour control. This applied to both a subcutaneous and a metastatic prostate cancer model, and indicates that the i.v. injected CAR T cells are capable of migrating to tumours in different locations. The data from the metastatic model is of particular relevance, as a clinical application of STEAP1 CAR T cells would be in metastatic patients. The results in the subcutaneous model were consistent between bioluminescence imaging and calliper measurements, which were applied in parallel for robust efficacy assessment. The antigen-specific effect of the STEAP1 CAR was demonstrated, as tumour control was not detected with a CD19 CAR T control that has an identical co-stimulatory domain and backbone. Furthermore, the experiments with the STEAP1 knockout 22RV1 line confirmed that the in vivo efficacy was dependent on STEAP1 tumour expression.

In contrast to hematological malignancies, recognition of solid tumours requires migration of immune cells from the blood into the tumours. In situations where the tumour is localized, different administration methods may be used to improve efficacy. For example, regional rather than systemic administration of CAR T cells might enhance efficacy. In other embodiments, systemic administration of the CAR T cells may be advantageous.

The pharmaceutical compositions may comprise a pharmaceutically effective dose of the immune cells herein. A pharmaceutically effective dose may for example be in the range of 1×10⁶ to 1×10¹⁰ immune cells expressing the CARs. A pharmaceutically effective dose may for example be in the range of 1×10⁶ to 1×10⁹ T cells expressing the CARs.

In one embodiment, a pharmaceutical composition is provided, which comprises a pharmaceutically effective dose of T cells or NK cells expressing a CAR as described herein, for use in treatment of STEAP1-positive cancer. Such compositions can be administered intravenously, STEAP1-positive cancer cells can be detected by anti-STEAP1 antibodies (e.g. X120.545.1.1 from ATCC PTA-5803).

In one particular embodiment, a method of treating a patient diagnosed with prostate cancer is provided, wherein the method comprises the steps:
a. obtaining a biological sample comprising cancer cells from the patient:
b. analysing whether the cancer cells express STEAP1; and
c. administering a pharmaceutical composition comprising a pharmaceutically effective dose of T cells or NK cells expressing any of the CARs disclosed herein if the cancer cells are STEAP1 positive.

The CAR-expressing immune effector cells may be administered in combination with one or more other therapeutic agents, which may include any other known cancer treatments, such as radiation therapy, chemotherapy, transplantation, immunotherapy, hormone therapy, photodynamic therapy, etc. The compositions may also be administered in combination with antibiotics or other therapeutic agents, including e.g. cytokines (e.g. IL-1, IL-2, IL-3, IL-4, IL-5, IL-6, IL-7, IL-8, IL-9, IL-10, IL-11, IL-12, IL-13, IL-15 and IL-17), growth factors, steroids, NSAIDs, DMARDs, anti-inflammatories, analgesics, chemotherapeutics (e.g. monomethyl auristatin E, fludarabine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, oxaliplatin, mitomycin, dacarbazine, procarbazine, etoposide, teniposide, campathecins, bleomycin, doxorubicin, idarubicin, daunorubicin, dactinomycin, plicamycin, mitoxantrone, L-asparaginase, 5-fluorouracil), radiotherapeutics, immune checkpoint inhibitors (e.g. Tremelimumab, Ipilimumab, Nivolumab, MK-3475, Urelumab, Bavituximab, MPDL3280A, MEDI4736), small molecule inhibitors or other active and ancillary agents.

The subject to be treated using the methods and cells of the present invention may be any species of mammal. For instance, the subject may be any species of domestic pet, such as a mouse, rat, gerbil, rabbit, guinea pig, hamster, cat or dog, or livestock, such as a goat, sheep, pig, cow or horse. In a preferred embodiment of the invention the subject is a primate, such as a monkey, gibbon, gorilla, orang-utang, chimpanzee or bonobo. However, in a preferred embodiment of the invention the subject is a human. Whilst the use of xenogeneic cells is not precluded, it is preferred that immune cells for administration are of the same species as the recipient. Thus, for human subjects it is preferred that the immune cells are human.

The present invention may be more fully understood from the non-limiting Examples below.

### EXAMPLES

### Example 1—Construction and Expression of CARs

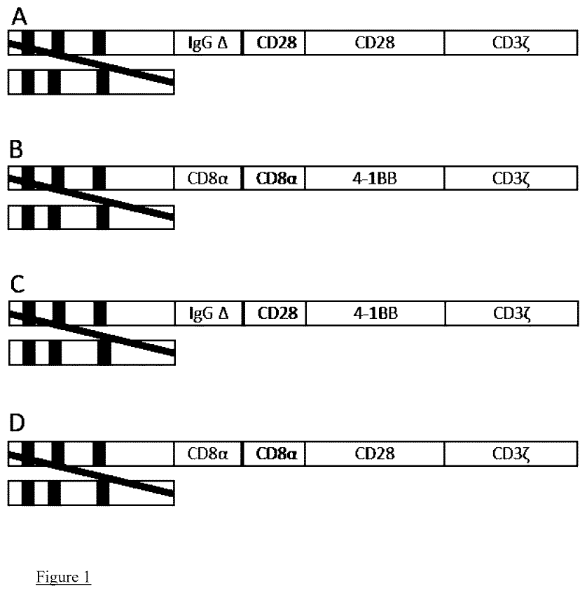

Nucleic acids encoding JK10 or JK11 were cloned into retroviral SFG vectors by standard methods in the art. The resulting vectors encoded either SEQ ID NO: 12 (for expression of JK10) or SEQ ID NO: 13 (for expression of JK11). Schematic structures of the JK10 and JK11 CARs are shown in FIG. 1. Both CARs were well expressed on the cell surface when T cells were transduced with the retroviral vectors (data not shown).

### Example 2—In Vitro Cytokine and CD107a Release

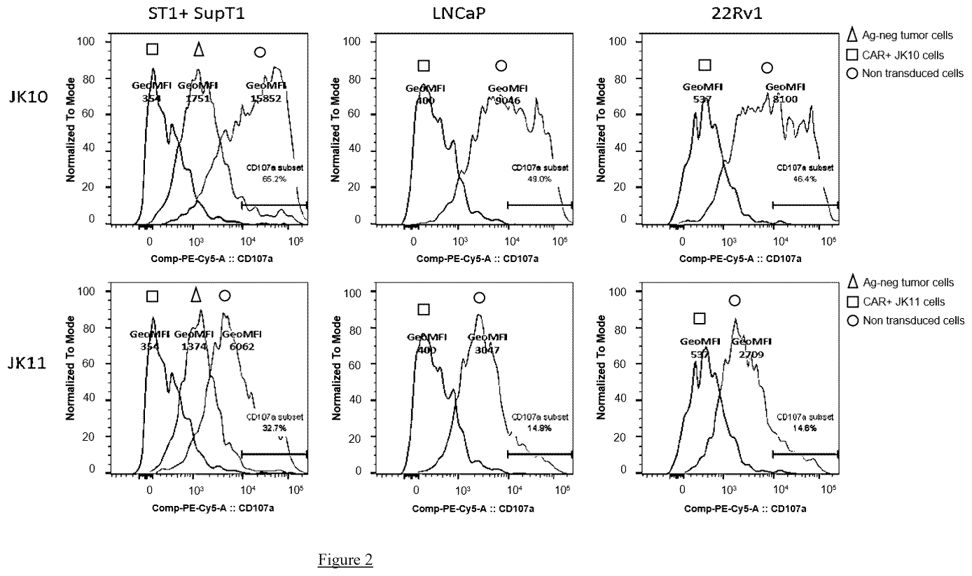

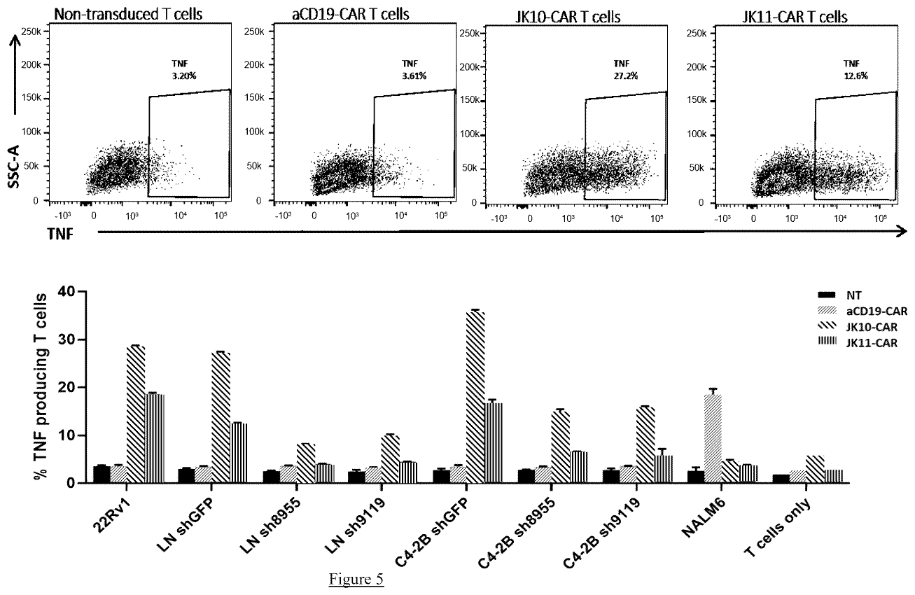

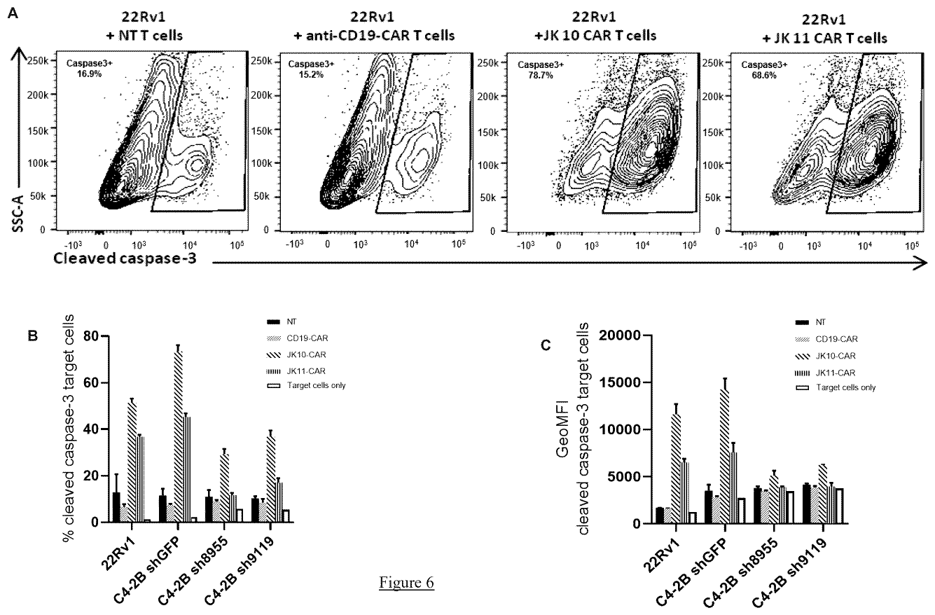

A composition comprising a mixture of CD4+T helper cells and CD8+ cytotoxic T cells was transduced with the vectors from Example 1 using X-tremeGENE HP DNA Transfection Reagent (Roche). Three different STEAP1-positive cell lines (3.6×10⁵/well) were co-incubated in a V-bottomed 96 well plate with 1.2×10⁵ T cells expressing either JK10 or JK11 (E:T=1:3). After 16 hours, IFNγ production in the total CD3+ T-cell population was analysed by flow cytometry. The data showed STEAP1-specific secretion of both IFNγ (FIG. 4), TNFα (FIG. 5) and CD107α (FIG. 2) for both JK10 and JK11 CAR T cells. The fraction of responding cells was generally higher for JK10 T cells than JK11 T cells. The STEAP1-specific response conferred by JK10 and JK11 was demonstrated in both CD4+ and CD8+ T cells (separate data for CD4+ and CD8+ T cells not shown). The fraction of cytokine secreting cells was higher among the cells expressing the CAR construct, as measured by the marker gene RQR8 (not shown).

In general, shRNA yields partial, but not complete, knock-down of the protein, in this case STEAP1. A low-level response remained for JK10 CAR T against shRNA controls, suggesting that JK10 CAR T cells may respond more than JK11 CAR T cells against targets with a low STEAP1 expression.

### Example 3—In Vitro Cytotoxicity

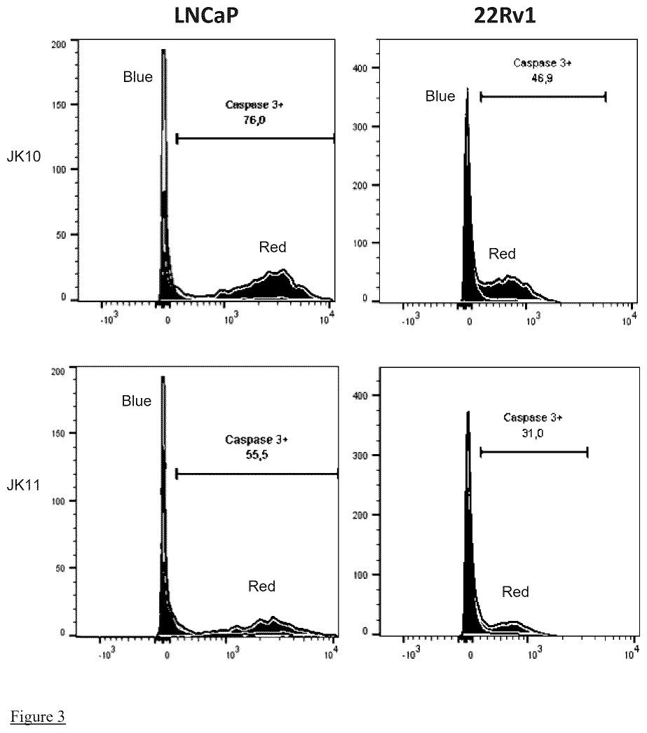

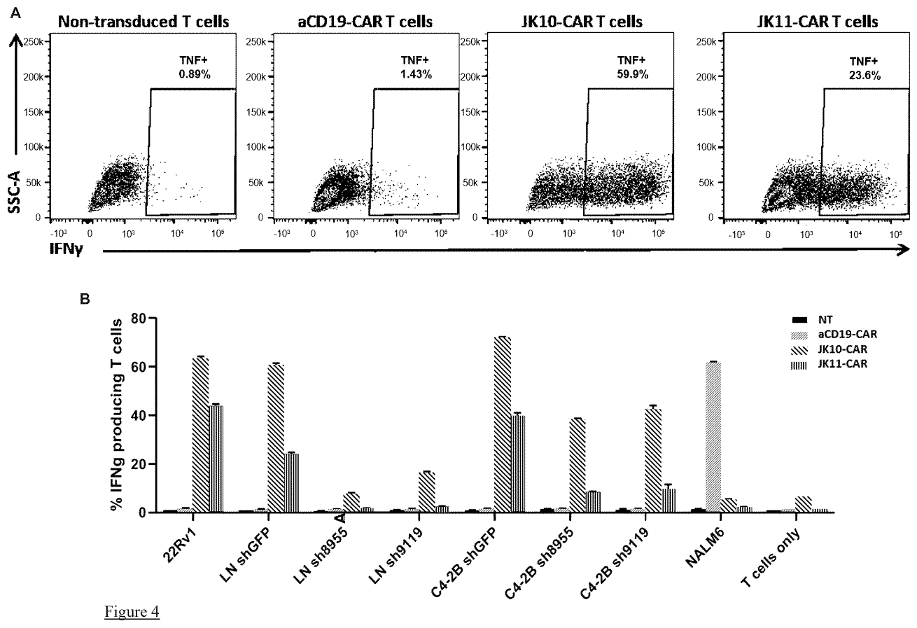

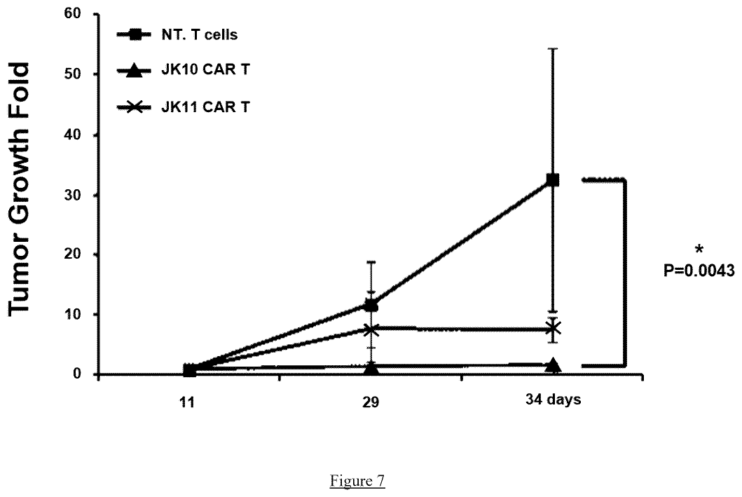

The killing capacity of if the CAR T cells was assessed both as cytolytic activity of the effector T cells (CD107a/TNF, FIGS. 2 and 5), by measurement of cell death in target cells (cleaved caspase 3, indicating apoptosis; FIGS. 3 and 6) and by the IncuCyte S3 real-time live cell analysis system. For the caspase assay, a composition comprising a mixture of T helper cells and cytotoxic T cells was transduced with the vectors from Example 1 using X-tremeGENE HP DNA Transfection Reagent (Roche). The STEAP1-positive (and knock-down) cell lines (5×10⁵/well) were co-incubated in a V-bottomed 96 well plate with 1×10⁵ T cells expressing either JK10 or JK11 (E:T=5:1). After 16 hours, caspase 3 cleavage in the target cell population was analysed by flow cytometry. The data demonstrated efficient and STEAP1-specific killing by the JK10 and JK11 CAR T cells, as compared to controls. A slightly stronger cytotoxic effect was observed for JK10 CAR T cells, as compared to JK11 CAR T cells.

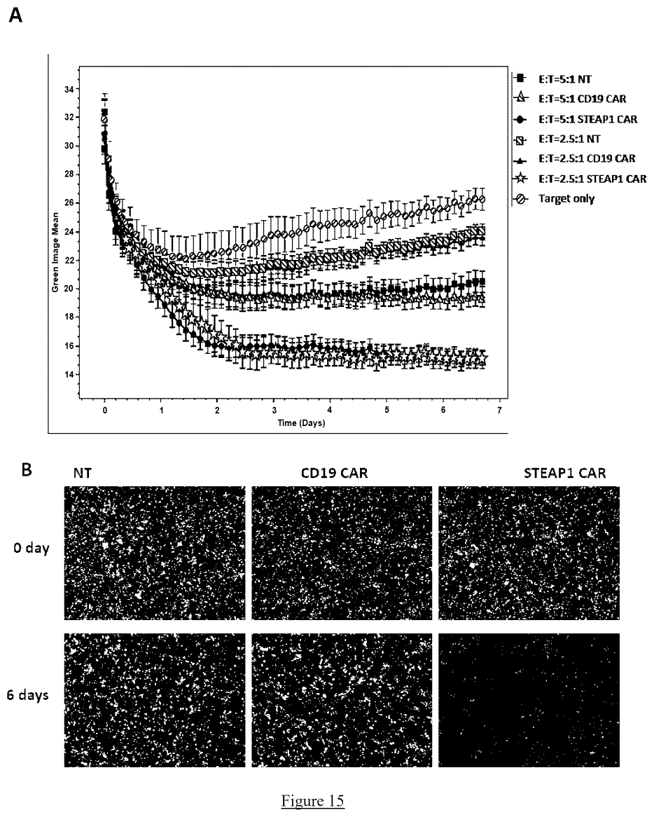

The IncuCyte S3 real-time live cell analysis system monitors cell death over time by the continuous imaging the GFP-labelled target cells. As shown in FIG. 15, the STEAP1 CAR T cells effectively eliminated the 22Rv1 cells, at both E:T ratio 5:1 and 2.5:1, while CD19 CAR T cell controls only gave limited non-specific effects, similar to NT T cells. The real-time data demonstrated ongoing killing of target cells over several days, only in those exposed to STEAP1 CAR T cells. After 6 days, nearly all target cells were dead (FIG. 15 A-B).

### Example 4—In Vivo Activity in Subcutaneous Tumor Model

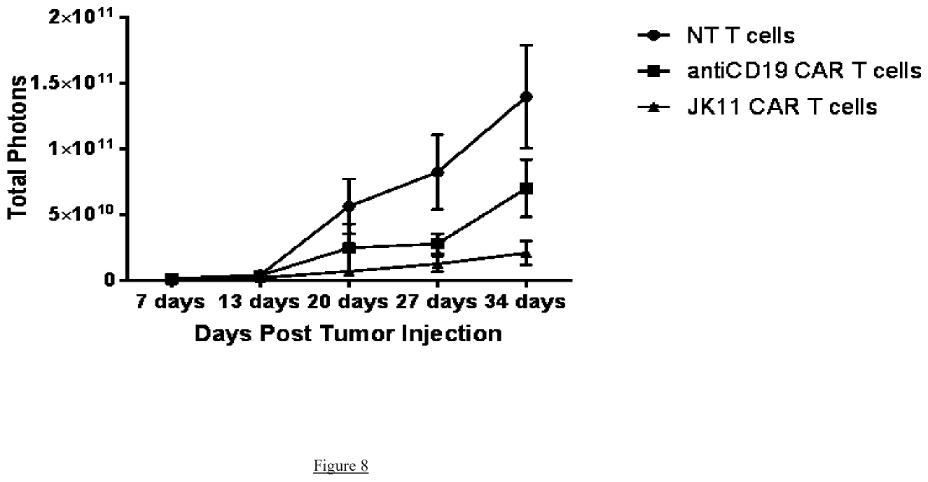

We injected 2×10⁶ luciferase transuded 22RV1 prostate cancer cells subcutaneously into NSG mice, in two separate experiments, shown in FIG. 7 and FIGS. 8-13, respectively. The experimental conditions are explained in the legends for each figure. As shown, a significant difference in tumour load developed between the control groups and the group of mice receiving T cells expressing JK10 (FIG. 7) or JK11 (FIGS. 8-12).

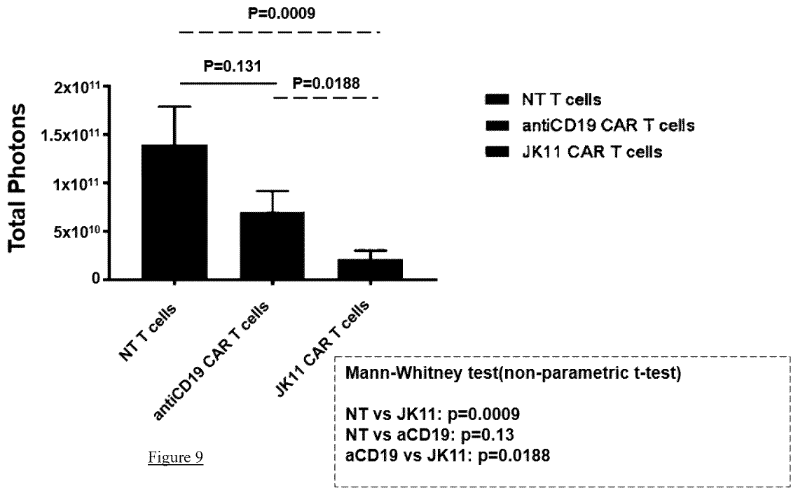

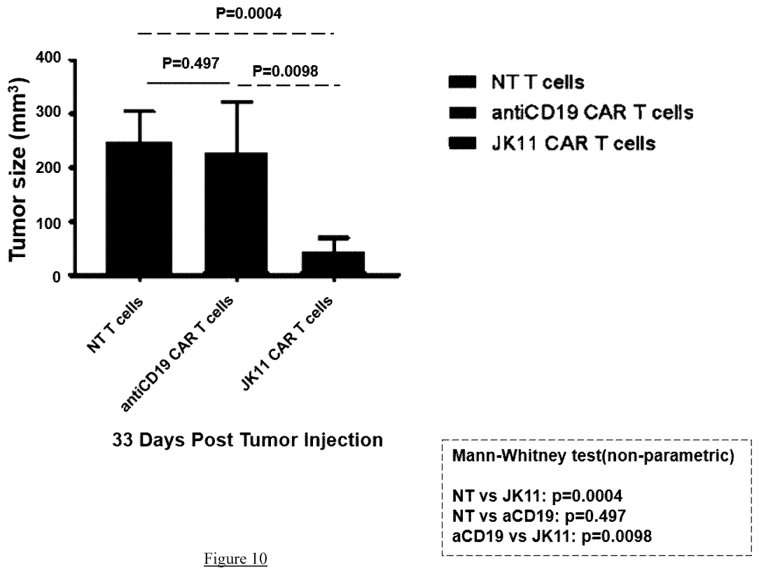

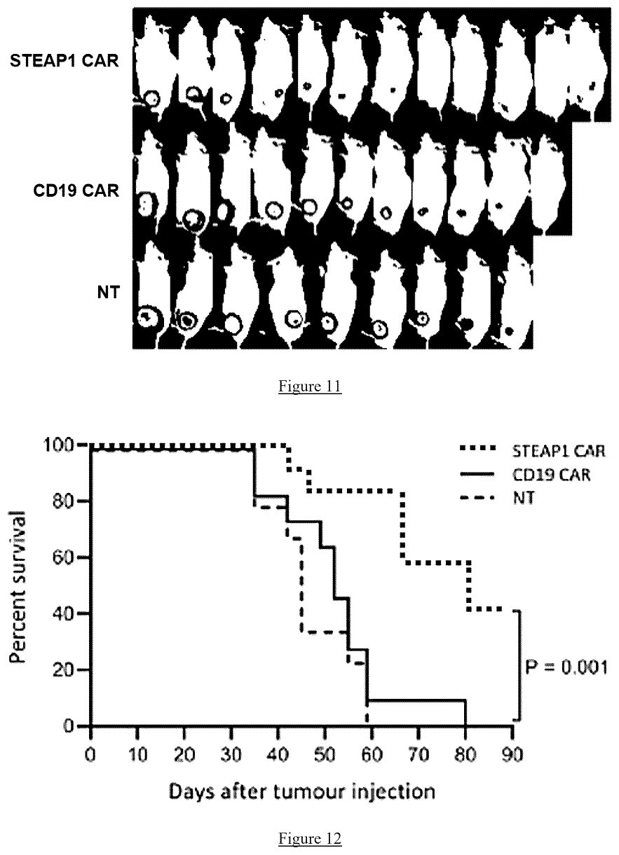

The tumours grew readily in the mice that were treated with CD19 CAR T cells or NT T cells, whereas the mice treated with JK11 STEAP1 CAR T cells displayed a substantial inhibition of tumour growth (FIG. 8). A statistically significant difference in tumour load developed between the mice in the STEAP1 CAR group and each of the control groups as measured by both bioluminescence imaging (FIG. 9) and calliper measurement (FIG. 10). At day 34, the tumours had disappeared in five out of twelve mice in the STEAP1 CAR treated group (FIG. 11). The tumours reoccurred around day 55 in four out of these five mice. By day 60, all mice treated with NT T cells had to be euthanized due to the large size of the tumours (FIG. 12), and only three CD19 CAR treated mice were still alive. The mice treated with STEAP1 CAR T cells had a significantly extended survival compared to both control groups, with 10 out of 12 mice still alive 60 days after tumour cell engraftment, and five of these mice still alive at the end of the experiment (day 85; FIG. 12).

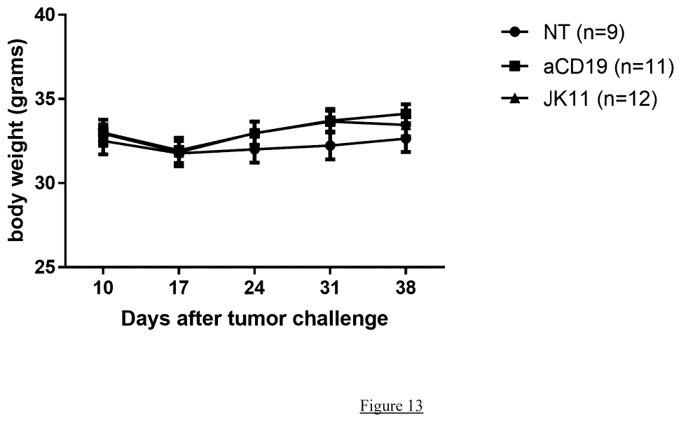

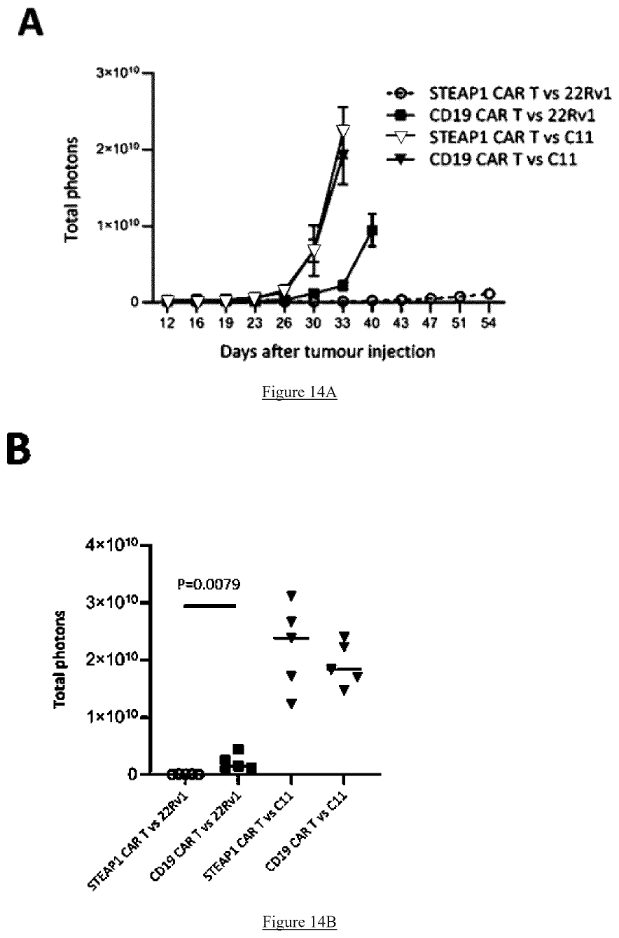

The results show that the CAR T cells are therapeutically effective against STEAP1+ cancer in vivo. No signs of adverse effects were observed. The toxicity assessment was based on monitoring of weight (FIG. 13) and of general well-being and activity of the mice.

### Example 5—Metastatic In Vivo Prostate Cancer Xenograft Studies

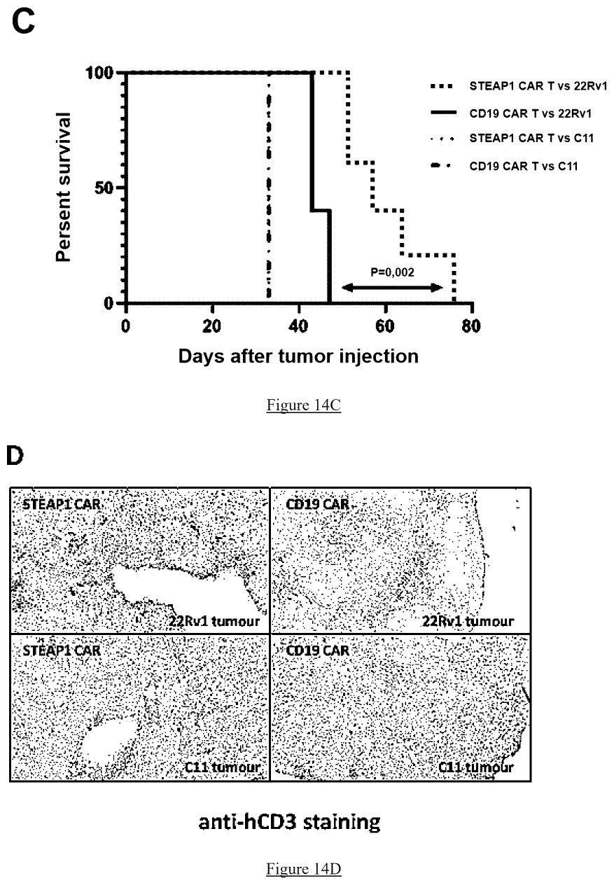

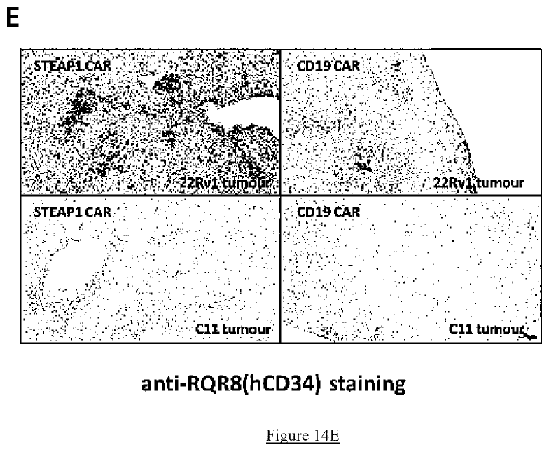

NXG (NOD.Cg-Prkdc^{scid}Il2^{tm1Wj1}/SzJ) (Janvier labs, Le Genest-Saint-Isle, France) immunodeficient mice were bred in-house. 6 to 8-week-old mice were injected i.v. with 10×10⁶ 22Rv1 cells (firefly luciferase transduced) or STEAP1 knockout 22Rv1 (clone C11) into the tail veins. When the bioluminescence signal was detectable (about 20 days post tumour injection), 1×10⁷ T cells per mouse were injected into the tail vein. A second T cell intravenous injection (1×10⁷ cells per mouse) was performed at the same time point for all mice in each experiment (7 days after the first injection, as indicated). 100 IU of rhIL-2 per gram body weight were injected i.p. twice per week. Tumour growth was monitored by bioluminescence imaging twice or once per week (Xenogen Spectrum system; Grantham, UK). Briefly, anesthetized mice were injected i.p. with 150 μg/g body weight of D-luciferin (Perkin Elmer Norway, Oslo, Norway) and imaged 12 min after luciferin injection. The results are visualized in FIG. 14.

LIST OF SEQUENCES
SEQ ID NO: 1
DVQVQESGPGLVKPSQSLSLTCTVTGYSITSDYAWNWIRQFPGNKLEWMG
YISNSGSTSYNPSLKSRISITRDTSKNQFFLQLISVTTEDTATYYCARER
NYDYDDYYYAMDYWGQGTSVTVSS
SEQ ID NO: 2
DIVMSQSPSSLAVSVGEKVTMSCKSSQSLLYRSNQKNYLAWYQQKPGQSP
KLLIYWASTRESGVPDRFTGSGSGTDFTLTISSVKAEDLAVYYCQQYYNY
PRTFGGGTKLEIK
SEQ ID NO: 3
SDPAEPKSPDKTHTCPPCPAPPVAGPSVFLFPPKPKDTLMIARTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTV
DKSRWQQGNVFSCSVMHEALHNHYTQKSLSLSPGKKDPK
SEQ ID NO: 4
FWVLVVVGGVLACYSLLVTVAFIIFW
SEQ ID NO: 5
VRSKRSRLLHSDYMNMTPRRPGPTRKHYQPYAPPRDFAAYR
SEQ ID NO: 6
SRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
SEQ ID NO: 7
GGGGSGGGGSGGGGSGGGGS
SEQ ID NO: 8
MVLILLWLFTAFPGILS
SEQ ID NO: 9
SDPTTTPAPRPPTPAPTIASQPLSLRPEACRPAAGGAVHTRGLDF
SEQ ID NO: 10
ACDIYIWAPLAGTCGVLLLSLVITLYC
SEQ ID NO: 11
KRGRKKLLYIFKQPFMRPVQTTQEEDGCSCRFPEEEEGGCEL
SEQ ID NO: 12
MVLILLWLFTAFPGILSDVQVQESGPGLVKPSQSLSLTCTVTGYSITSDY
AWNWIRQFPGNKLEWMGYISNSGSTSYNPSLKSRISITRDTSKNQFFLQL
ISVTTEDTATYYCARERNYDYDDYYYAMDYWGQGTSVTVSSGGGGGGGGS
GGGGSGGGGSDIVMSQSPSSLAVSVGEKVTMSCKSSQSLLYRSNQKNYLA
WYQQKPGQSPKLLIYWASTRESGVPDRFTGSGSGTDFTLTISSVKAEDLA
VYYCQQYYNYPRTFGGGTKLEIKSDPAEPKSPDKTHTCPPCPAPPVAGPS
VFLFPPKPKDTLMIARTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQK
SLSLSPGKKDPKFWVLVVVGGVLACYSLLVTVAFIIFWVRSKRSRLLHSD
YMNMTPRRPGPTRKHYQPYAPPRDFAAYRSRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 13
MVLILLWLFTAFPGILSDVQVQESGPGLVKPSQSLSLTCTVTGYSITSDY
AWNWIRQFPGNKLEWMGYISNSGSTSYNPSLKSRISITRDTSKNQFFLQL
ISVTTEDTATYYCARERNYDYDDYYYAMDYWGQGTSVTVSSGGGGSGGGG
SGGGGSGGGGSDIVMSQSPSSLAVSVGEKVTMSCKSSQSLLYRSNQKNYL
AWYQQKPGQSPKLLIYWASTRESGVPDRFTGSGSGTDFTLTISSVKAEDL
AVYYCQQYYNYPRTFGGGTKLEIKSDPTTTPAPRPPTPAPTIASQPLSLR
PEACRPAAGGAVHTRGLDFACDIYIWAPLAGTCGVLLLSLVITLYCKRGR
KKLLYIFKQPFMRPVQTTQEEDGCSCRFPEEEEGGCELSRVKFSRSADAP
AYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R
SEQ ID NO: 14
GYSITSDYAWN
SEQ ID NO: 15
GYISNSGSTSYNPSLKSR
SEQ ID NO: 16
ERNYDYDDYYYAMDY
SEQ ID NO: 17
KSSQSLLYRSNQKNYLA
SEQ ID NO: 18
WASTRES
SEQ ID NO: 19
QQYYNYPRT
SEQ ID NO: 20
ERNYDYEDYYYAMDY
SEQ ID NO: 21
ERNYDYDEYYYAMDY
SEQ ID NO: 22
ERNYDYEEYYYAMDY
SEQ ID NO: 23
DVQVQESGPGLVKPSQSLSLTCTVTGYSITSDYAWNWIRQFPGNKLEWMG
YISNSGSTSYNPSLKSRISITRDTSKNQFFLQLISVTTEDTATYYCARER
NYDYDDYYYAMDYWGQGTSVTVSSGGGGSGGGGSGGGGSGGGGSDIVMSQ
SPSSLAVSVGEKVTMSCKSSQSLLYRSNQKNYLAWYQQKPGQSPKLLIYW
ASTRESGVPDRFTGSGSGTDFTLTISSVKAEDLAVYYCQQYYNYPRTFGG
GTKLEIK
SEQ ID NO: 24
DVQVQESGPGLVKPSQSLSLTCTVTGYSITSDYAWNWIRQFPGNKLEWMG
YISNSGSTSYNPSLKSRISITRDTSKNQFFLQLISVTTEDTATYYCARER
NYDYDDYYYAMDYWGQGTSVTVSSGGGGSGGGGSGGGGSGGGGSDIVMSQ
SPSSLAVSVGEKVTMSCKSSQSLLYRSNQKNYLAWYQQKPGQSPKLLIYW
ASTRESGVPDRFTGSGSGTDFTLTISSVKAEDLAVYYCQQYYNYPRTFGG
GTKLEIKSDPAEPKSPDKTHTCPPCPAPPVAGPSVFLFPPKPKDTLMIAR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFF
LYSKLTVDKSRWQQGNVFSCSVMHEALHNHYTQKSLSLSPGKKDPKFWVL
VVVGGVLACYSLLVTVAFIIFWVRSKRSRLLHSDYMNMTPRRPGPTRKHY
QPYAPPRDFAAYRSRVKFSRSADAPAYQQGQNQLYNELNLGRREEYDVLD
KRRGRDPEMGGKPRRKNPQEGLYNELQKDKMABAYSEIGMKGERRRGKGH
DGLYQGLSTATKDTYDALHMQALPPR
SEQ ID NO: 25
DVQVQESGPGLVKPSQSLSLTCTVTGYSITSDYAWNWIRQFPGNKLEWMG
YISNSGSTSYNPSLKSRISITRDTSKNQFFLQLISVTTEDTATYYCARER
NYDYDDYYYAMDYWGQGTSVTVSSGGGGSGGGGSGGGGSGGGGSDIVMSQ
SPSSLAVSVGEKVTMSCKSSQSLLYRSNQKNYLAWYQQKPGQSPKLLIYW
ASTRESGVPDRFTGSGSGTDFTLTISSVKAEDLAVYYCQQYYNYPRTFGG
GTKLEIKSDPTTTPAPRPPTPAPTIASQPLSLRPEACRPAAGGAVHTRGL
DFACDIYIWAPLAGTCGVLLLSLVITLYCKRGRKKLLYIFKQPFMRPVQT
TQEEDGCSCRFPEEEEGGCELSRVKFSRSADAPAYQQGQNQLYNELNLGR
REEYDVLDKRRGRDPEMGGKPRRKNPQEGLYNELQKDKMAEAYSEIGMKG
ERRRGKGHDGLYQGLSTATKDTYDALHMQALPPR
SEQ ID NO: 26
DVQVQESGPGLVKPSQSLSLTCTVTGYSITSDYAWNWIRQFPGNKLEWMG
YISNSGSTSYNPSLKSRISITRDTSKNQFFLQLISVTTEDTATYYCARER
NYDYDDYYYAMDYWGQGTSVTVSSGGGGSGGGGSGGGGSGGGGSDIVMSQ
SPSSLAVSVGEKVTMSCKSSQSLLYRSNQKNYLAWYQQKPGQSPKLLIYW
ASTRESGVPDRFTGSGSGTDFTLTISSVKAEDLAVYYCQQYYNYPRTFGG
GTKLEIKSDPAEPKSPDKTHTCPPCPAPPVAGPSVFLFPPKPKDTLMIAR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFF
LYSKLTVDKSRWQQGNVFSCSVMHEALHNHYTQKSLSLSPGKKDPKFWVL
VVVGGVLACYSLLVTVAFIIFWKRGRKKLLYIFKQPFMRPVQTTQEEDGC
SCRFPEEEEGGCELSRVKFSRSADAPAYQQGQNQLYNELNLGRREEYDVL
DKRRGRDPEMGGKPRRKNPQEGLYNELQKDKMAEAYSEIGMKGERRRGKG
HDGLYQGLSTATKDTYDALHMQALPPR
SEQ ID NO: 27
DVQVQESGPGLVKPSQSLSLTCTVTGYSITSDYAWNWIRQFPGNKLEWMG
YISNSGSTSYNPSLKSRISITRDTSKNQFFLQLISVTTEDTATYYCARER
NYDYDDYYYAMDYWGQGTSVTVSSGGGGSGGGGSGGGGSGGGGSDIVMSQ
SPSSLAVSVGEKVTMSCKSSQSLLYRSNQKNYLAWYQQKPGQSPKLLIYW
ASTRESGVPDRFTGSGSGTDFTLTISSVKAEDLAVYYCQQYYNYPRTFGG
GTKLEIKSDPTTTPAPRPPTPAPTIASQPLSLRPEACRPAAGGAVHTRGL
DFACDIYIWAPLAGTCGVLLLSLVITLYCVRSKRSRLLHSDYMNMTPRRP
GPTRKHYQPYAPPRDFAAYRSRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR
SEQ ID NO: 28
MVLILLWLFTAFPGILSDVQVQESGPGLVKPSQSLSLTCTVTGYSITSDY
AWNWIRQFPGNKLEWMGYISNSGSTSYNPSLKSRISITRDTSKNQFFLQL
ISVTTEDTATYYCARERNYDYDDYYYAMDYWGQGTSVTVSSGGGGSGGGG
SGGGGSGGGGSDIVMSQSPSSLAVSVGEKVTMSCKSSQSLLYRSNQKNYL
AWYQQKPGQSPKLLIYWASTRESGVPDRFTGSGSGTDFTLTISSVKAEDL
AVYYCQQYYNYPRTFGGGTKLEIKSDPAEPKSPDKTHTCPPCPAPPVAGP
SVFLFPPKPKDTLMIARTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQ
KSLSLSPGKKDPKFWVLVVVGGVLACYSLLVTVAFIIFWKRGRKKLLYIF
KQPFMRPVQTTQEEDGCSCRFPEEEEGGCELSRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR
SEQ ID NO: 29
MVLILLWLFTAFPGILSDVQVQESGPGLVKPSQSLSLTCTVTGYSITSDY
AWNWIRQFPGNKLEWMGYISNSGSTSYNPSLKSRISITRDTSKNQFFLQL
ISVTTEDTATYYCARERNYDYDDYYYAMDYWGQGTSVTVSSGGGGSGGGG
SGGGGSGGGGSDIVMSQSPSSLAVSVGEKVTMSCKSSQSLLYRSNQKNYL
AWYQQKPGQSPKLLIYWASTRESGVPDRFTGSGSGTDFTLTISSVKAEDL
AVYYCQQYYNYPRTFGGGTKLEIKSDPTTTPAPRPPTPAPTIASQPLSLR
PEACRPAAGGAVHTRGLDFACDIYIWAPLAGTCGVLLLSLVITLYCVRSK
RSRLLHSDYMNMTPRRPGPTRKHYQPYAPPRDFAAYRSRVKFSRSADAPA
YQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
SEQ ID NO: 30
SDPAEPKSPDKTHTCPPCPAPELLGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLT
VDKSRWQQGNVFSCSVMHEALHNHYTQKSLSLSPGKKDPK

1. A nucleic acid molecule encoding a chimeric antigen receptor (CAR) directed against STEAP1, wherein said CAR comprises an antigen-binding domain comprising a V_{H} region and a V_{L} region, each comprising three CDR sequences, wherein:
a) CDRs 1, 2 and 3 of the V_{H} region have the amino acid sequences of SEQ ID NOs: 14, 15 and 16 respectively; and
b) CDRs 1, 2 and 3 of the V_{H} region have the amino acid sequences of SEQ ID NOs: 17, 18 and 19 respectively; and
wherein one or more of said CDR sequences may optionally be modified by substitution, addition and/or deletion of 1 to 3 amino acids.

2. The nucleic acid molecule of claim 1, wherein:
a) CDRs 1 and 2 of the V_{H} region have the amino acid sequences of SEQ ID NOs: 14 and 15 respectively;
b) CDRs 1, 2 and 3 of the V_{L} region have the amino acid sequences of SEQ ID NOs: 17, 18 and 19 respectively; and
c) CDR3 of the V_{H} region has the amino acid sequence of SEQ ID NO: 16, or an amino acid sequence comprising up to 2 amino acid substitutions relative to SEQ ID NO: 16.

3. The nucleic acid molecule of claim 2, wherein:
a) CDRs 1 and 2 of the V_{H} region have the amino acid sequences of SEQ ID NOs: 14 and 15 respectively;
b) CDRs 1, 2 and 3 of the V_{L} region have the amino acid sequences of SEQ ID NOs: 17, 18 and 19 respectively; and
c) CDR3 of the V_{H} region has the amino acid sequence of SEQ ID NO: 16.

4. The nucleic acid molecule of claim 2, wherein:
a) CDRs 1 and 2 of the V_{H} region have the amino acid sequences of SEQ ID NOs: 14 and 15 respectively;
b) CDRs 1, 2 and 3 of the V_{L} region have the amino acid sequences of SEQ ID NOs: 17, 18 and 19 respectively; and
c) CDR3 of the V_{H} sequence has the amino acid sequence of SEQ ID NO: 20, SEQ ID NO: 21 or SEQ ID NO: 22.

5. The nucleic acid molecule of any one of claims 1 to 3, wherein the V_{H} region comprises the amino acid sequence of SEQ ID NO: 1, or an amino acid sequence with at least 80% sequence identity thereto; and
the V_{L} region comprises the amino acid sequence of SEQ ID NO: 2, or an amino acid sequence with at least 80% sequence identity thereto.

6. The nucleic acid molecule of claim 5, wherein in the encoded CAR the antigen binding domain is an scFv, wherein the V_{H} region and the V_{L} region are joined by a peptide linker.

7. The nucleic acid molecule of claim 6, wherein in the encoded CAR the scFv comprises the amino acid sequence set forth in SEQ ID NO: 23, or an amino acid sequence having at least 90% sequence identity thereto.

8. The nucleic acid molecule of any one of claims 1 to 7, wherein the encoded CAR comprises a hinge domain selected from:
(a) the IgGΔ hinge domain, comprising or consisting of the amino acid sequence of SEQ ID NO: 3, or an amino acid sequence having at least 95% sequence identity thereto; or
(b) the CD8α hinge domain, comprising or consisting of the amino acid sequence of SEQ ID NO: 9, or an amino acid sequence having at least 95% sequence identity thereto.

9. The nucleic acid molecule of any one of claims 1 to 8, wherein the encoded CAR comprises a transmembrane domain selected from:
(a) the CD28 transmembrane domain, comprising or consisting of the amino acid sequence of SEQ ID NO: 4, or an amino acid sequence having at least 95% sequence identity thereto; or
(b) the CD8α transmembrane domain, comprising or consisting of the amino acid sequence of SEQ ID NO: 10, or an amino acid sequence having at least 95% sequence identity thereto.

10. The nucleic acid molecule of any one of claims 1 to 9, wherein the encoded CAR comprises an intracellular signalling domain comprising the CD3ζ signalling domain, comprising or consisting of the amino acid sequence of SEQ ID NO: 6, or an amino acid sequence having at least 95% sequence identity thereto.

11. The nucleic acid molecule of claim 10, wherein the intracellular signalling domain further comprises a costimulatory domain.

12. The nucleic acid molecule of claim 11, wherein the costimulatory domain is selected from:
(a) the CD28 costimulatory domain, comprising or consisting of the amino acid sequence of SEQ ID NO: 5, or an amino acid sequence having at least 95% sequence identity thereto; or
(b) the 4-1BB costimulatory domain, comprising or consisting of the amino acid sequence of SEQ ID NO: 11, or an amino acid sequence having at least 95% sequence identity thereto.

13. The nucleic acid molecule of claim 1, wherein the encoded CAR comprises the amino acid sequence of SEQ ID NO: 24, or an amino acid sequence having at least 95% sequence identity thereto.

14. The nucleic acid molecule of claim 1, wherein the encoded CAR comprises the amino acid sequence of SEQ ID NO: 25, or an amino acid sequence having at least 95% sequence identity thereto.

15. A vector, preferably an expression vector or a cloning vector, comprising the nucleic acid molecule of any one of claims 1 to 14.

16. An immune effector cell comprising the nucleic acid molecule of any one of claims 1 to 14 or the vector of claim 15, wherein the immune effector cell expresses a CAR as defined in any one of claims 1 to 14 at its surface.

17. The immune effector cell of claim 16, wherein the immune effector cell is a human T cell or a human NK cell.

18. A composition comprising the immune effector cell of claim 16 or 17 and a pharmaceutically acceptable carrier or excipient.

19. A CAR encoded by a nucleic acid as defined in any one of claims 1 to 14.

20. The immune effector cell of claim 16 or 17, the composition of claim 18, or the CAR of claim 19, for use in therapy.

21. The immune effector cell of claim 16 or 17, the composition of claim 18, or the CAR of claim 19, for use in the treatment of STEAP1-positive prostate cancer.

22. The immune effector cell, composition or CAR for use according to claim 21, wherein said prostate cancer is metastatic prostate cancer.

23. The immune effector cell, composition or CAR for use according to claim 21, wherein said prostate cancer is castration-resistant.


SEQUENCE LISTING (30 sequences)

SEQ ID NO: 1 - protein, 124 aa, synthetic construct
  source 1..124: note = Antigen-binding domain VH
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSS

SEQ ID NO: 2 - protein, 113 aa, synthetic construct
  source 1..113: note = Antigen-binding domain VL
DIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIK

SEQ ID NO: 3 - protein, 239 aa, synthetic construct
  source 1..239: note = IgGdelta hinge
SDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 4 - protein, 26 aa, synthetic construct
  source 1..26: note = CD28 transmembrane domain
FWVLVVVGGVLACYSLLVTVAFIIFW

SEQ ID NO: 5 - protein, 41 aa, synthetic construct
  source 1..41: note = CD28 costimulatory domain
VRSKRSRLLHSDYMNMTPRRPGPTRKHYQPYAPPRDFAAYR

SEQ ID NO: 6 - protein, 113 aa, synthetic construct
  source 1..113: note = CD3zeta signaling domain
SRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 20 aa, synthetic construct
  source 1..20: note = Linker of variant VHCDR3 sequences
GGGGSGGGGSGGGGSGGGGS

SEQ ID NO: 8 - protein, 17 aa, synthetic construct
  source 1..17: note = Leader peptide
MVLILLWLFTAFPGILS

SEQ ID NO: 9 - protein, 45 aa, Homo sapiens
  source 1..45: note = Human CD8alpha hinge
SDPTTTPAPRPPTPAPTIASQPLSLRPEACRPAAGGAVHTRGLDF

SEQ ID NO: 10 - protein, 27 aa, synthetic construct
  source 1..27: note = CD8alpha transmembrane domain
ACDIYIWAPLAGTCGVLLLSLVITLYC

SEQ ID NO: 11 - protein, 42 aa, synthetic construct
  source 1..42: note = 4-1BB costimulatory domain
KRGRKKLLYIFKQPFMRPVQTTQEEDGCSCRFPEEEEGGCEL

SEQ ID NO: 12 - protein, 693 aa, synthetic construct
  source 1..693: note = JK10 CAR
MVLILLWLFTAFPGILSDVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 501 aa, synthetic construct
  source 1..501: note = JK11 CAR
MVLILLWLFTAFPGILSDVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 11 aa, synthetic construct
  source 1..11: note = VH comprising CDR1
GYSITSDYAWN

SEQ ID NO: 15 - protein, 18 aa, synthetic construct
  source 1..18: note = VH comprising CDR2
GYISNSGSTSYNPSLKSR

SEQ ID NO: 16 - protein, 15 aa, synthetic construct
  source 1..15: note = VH comprising CDR3
ERNYDYDDYYYAMDY

SEQ ID NO: 17 - protein, 17 aa, synthetic construct
  source 1..17: note = VL comprising CDR1
KSSQSLLYRSNQKNYLA

SEQ ID NO: 18 - protein, 7 aa, synthetic construct
  source 1..7: note = VL comprising CDR2
WASTRES

SEQ ID NO: 19 - protein, 9 aa, synthetic construct
  source 1..9: note = VL comprising CDR3
QQYYNYPRT

SEQ ID NO: 20 - protein, 15 aa, synthetic construct
  source 1..15: note = Variant VHCDR3
ERNYDYEDYYYAMDY

SEQ ID NO: 21 - protein, 15 aa, synthetic construct
  source 1..15: note = Variant VHCDR3
ERNYDYDEYYYAMDY

SEQ ID NO: 22 - protein, 15 aa, synthetic construct
  source 1..15: note = Variant VHCDR3
ERNYDYEEYYYAMDY

SEQ ID NO: 23 - protein, 257 aa, synthetic construct
  source 1..257: note = CAR scFv
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIK

SEQ ID NO: 24 - protein, 676 aa, synthetic construct
  source 1..676: note = JK10
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 484 aa, synthetic construct
  source 1..484: note = JK11
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 677 aa, synthetic construct
  source 1..677: note = JK15
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 483 aa, synthetic construct
  source 1..483: note = JK16
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPTTTPAPRPPTPAPTIASQPLSLRPEACRPAAGGAVHTRGLDFACDIYIWAPLAGTCGVLLLSLVITLYCVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 694 aa, synthetic construct
  source 1..694: note = JK15 CAR leader sequence
MVLILLWLFTAFPGILSDVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 500 aa, synthetic construct
  source 1..500: note = JK16 CAR leader sequence
MVLILLWLFTAFPGILSDVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPTTTPAPRPPTPAPTIASQPLSLRPEACRPAAGGAVHTRGLDFACDIYIWAPLAGTCGVLLLSLVITLYCVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 240 aa, Homo sapiens
  source 1..240: note = IgG hinge domain
SDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK